(19) United States
(12) Patent Application Publication
(10) Pub. No.: US 2024/0366618 A1
(43) Pub. Date: Nov. 7, 2024

(54) CGRP Antagonists for Treating Trigeminal Neuralgia

(71) Applicant: Pfizer Ireland Pharmaceuticals, Cork (IE)

(72) Inventors: Vladimir Coric, New Haven, CT (US); Robert Croop, New Haven, CT (US); Melissa Beiner, New Haven, CT (US)

(73) Assignee: Pfizer Ireland Pharmaceuticals, Cork (IE)

(21) Appl. No.: 18/700,075

(22) PCT Filed: Oct. 10, 2022
(86) PCT No.: PCT/US2022/046140

§ 371 Date: Apr. 10, 2024

Related U.S. Application Data
(60) Provisional application No. 63/254,194, filed on Oct. 11, 2021.

(51) Int. Cl. A61K 31/537; A61K 31/4545; A61K 31/496; A61K 31/517; A61P 25/02

(52) U.S. Cl. CPC A61K 31/537; A61K 31/4545; A61K 31/496; A61K 31/517; A61P 25/02

(57) ABSTRACT

Provided for are methods of treating neuralgia, including trigeminal neuralgia with one or more CGRP receptor antagonists and/or pharmaceutical compositions thereof. Such methods are useful for treating, ameliorating, alleviating, providing for prophylaxis or prevention of, halting the progression of, and/or reducing the risk of neuralgia, including trigeminal neuralgia in a mammalian subject, such as a human. In particular, CGRP receptor antagonists such as those according to Formula I, Formula II, and/or Formula III as described herein are useful in methods of treating neuralgia, including trigeminal neuralgia in a subject in need thereof.

### FIELD OF THE DISCLOSURE

Provided for are methods of treating neuralgia, including trigeminal neuralgia, with one or more calcitonin gene-related peptide (CGRP) receptor antagonists and/or pharmaceutical compositions thereof. Such methods are useful for treating, ameliorating, alleviating, providing for prophylaxis or prevention of, halting the progression of, and/or reducing the risk of neuralgia, including trigeminal neuralgia in a mammalian subject, such as a human. In particular, CGRP receptor antagonists such as those according to Formula I, Formula II, and/or Formula III as described herein are useful in methods of treating neuralgia, including trigeminal neuralgia, in a subject in need thereof.

### BACKGROUND OF THE DISCLOSURE

Neuralgia is generally pain caused by irritated or damaged nerves and may occur in any part of the body. Trigeminal neuralgia is the most common facial pain syndrome, affecting approximately 4-13 people per 100,000 annually. (Jones M R, Urits I, Ehrhardt K P, et al. A Comprehensive Review of Trigeminal Neuralgia. Curr Pain Headache Rep. 2019; 23(10):74). The classic form of trigeminal neuralgia is characterized by episodes of severe stabbing or shock-like pain, lasting seconds to minutes, along the distribution of the trigeminal nerve. In contrast, atypical trigeminal neuralgia presents as constant, burning pain along the same nerve distribution and tends to be more treatment-resistant than the classical form. Trigeminal neuralgia is thought to be caused by insult to the trigeminal nerve, such as neurovascular compression, mass effect from a local tumor, or direct traumatic or ischemic nerve injury. While several medical and surgical therapies are available for trigeminal neuralgia, long-term treatment efficacy is limited. Thus, there is a need for novel therapeutics for trigeminal neuralgia, including its atypical variant.

First-line treatment of trigeminal neuralgia consists of medical therapy with carbamazepine and oxcarbazepine. While response rates with initial therapy are approximately 70% for patients with classic trigeminal neuralgia, treatment efficacy with these medications wanes over time and there is limited evidence for alternative pharmacotherapies. Thus, patients who either do not achieve pain relief or cannot tolerate initial medical therapy often undergo neurosurgical intervention, such as rhizotomy or microvascular decompression. While immediate relief after surgical treatment ranges from 80-90%, only 50-70% of patients have persistent long-term improvement.

Furthermore, treatment outcomes are generally worse for patients with atypical trigeminal neuralgia pain, both with medical and surgical therapy. This may be due to underlying differences in pathophysiology between the classic and atypical subtypes, as suggested by differences in demyelination noted in a study utilizing diffusion tensor imaging to characterize the disease. (Willsey M S, Collins K L, Conrad E C, Chubb H A, Patil P G. Diffusion tensor imaging reveals microstructural differences between subtypes of trigeminal neuralgia. J Neurosurg. 2019; 133(2):573-579)

Additionally, adverse effects due to current medical and surgical treatment options are significant. Over 40% of patients undergoing traditional medical management, which can include anticonvulsants, muscle relaxants, and opiates, experience adverse effects, including nausea, fatigue and vertigo. (Jones et al. & Parascandolo E, Levinson K, Rizzoli P, Sharon R. Efficacy of Erenumab in the Treatment of Trigeminal Neuralgia: A Retrospective Case Series. Neurol Clin Pract. 2021; 11(3):227-231) Surgical procedures are also associated with a number of complications, including cranial nerve palsy, hearing loss, and facial hypesthesia, and increased postoperative pain. While these complications are rare, post-operative dysesthesias are especially difficult to manage. Given these considerations, there is a great need to develop novel therapeutics to treat pain associated with trigeminal neuralgia.

CGRP is a naturally occurring 37-amino-acid peptide first identified in 1982 (Amara, S. G. et al, Science 1982, 298, 240-244). Two forms of the peptide are expressed (αCGRP and βCGRP) which differ by one and three amino acids in rats and humans, respectively. The peptide is widely distributed in both the peripheral (PNS) and central nervous system (CNS), principally localized in sensory afferent and central neurons, and displays a number of biological effects, including vasodilation. CGRP is abundant in the trigeminal ganglion and implicated in several pain syndromes, including cluster headaches and migraine. One of the primary mechanisms of action of CGRP is sensitization of trigeminal afferent neurons, facilitating nociceptive transmission.

When released from the cell, CGRP binds to specific cell surface G protein-coupled receptors and exerts its biological action predominantly by activation of intracellular adenylate cyclase (Poyner, D. R. et al, Br J Pharmacol 1992, 105, 441-7; Van Valen, F. et al, Neurosci Lett 1990, 119, 195-8.). Two classes of CGRP receptors, CGRP1 and CGRP2, have been proposed based on the antagonist properties of the peptide fragment CGRP(8-37) and the ability of linear analogues of CGRP to activate CGRP2 receptors (Juaneda, C. et al. TiPS 2000, 21, 432-438). However, there is lack of molecular evidence for the CGRP2 receptor (Brain, S. D. et al, TiPS 2002, 23, 51-53). The CGRP1 receptor has three components: (i) a 7 transmembrane calcitonin receptor-like receptor (CRLR); (ii) the single transmembrane receptor activity modifying protein type one (RAMP1); and (iii) the intracellular receptor component protein (RCP) (Evans B. N. et al., J Biol. Chem. 2000, 275, 31438-43). RAMP 1 is required for transport of CRLR to the plasma membrane and for ligand binding to the CGRP-receptor (McLatchie, L. M. et al, Nature 1998, 393, 333-339). RCP is required for signal transduction (Evans B. N. et al., J Biol. Chem. 2000, 275, 31438-43). There are known species-specific differences in binding of small molecule antagonists to the CGRP-receptor with typically greater affinity seen for antagonism of the human receptor than for other species (Brain, S. D. et al, TiPS 2002, 23, 51-53). The amino acid sequence of RAMP1 determines the species selectivity, in particular, the amino acid residue Trp74 is responsible for the phenotype of the human receptor (Mallee et al. J Biol Chem 2002, 277, 14294-8).

Inhibitors at the receptor level to CGRP are postulated herein to be useful in pathophysiologic conditions where excessive CGRP receptor activation has occurred, such as in trigeminal neuralgia. Some further examples include neurogenic vasodilation, neurogenic inflammation, migraine, cluster headache and other headaches, thermal injury, circulatory shock, and asthma, as well as various skin conditions having a neurogenic component including psoriasis, redness, rosacea, discrete erythema, flushing, menopausal flushing, rash, and hyperseborrhoea. CGRP antagonists or inhibitors are also contemplated to be useful in subjective sensations of itching, pruritus, sensations of burning or heating, sensations of stinging, tingling, discomfort, tightness, and other such sensations having either a neurogenic component or a neurogenic origin.

Prior studies have also suggested that CGRP is implicated specifically in trigeminal neuralgia pain. For example, animal models of trigeminal neuropathic pain have demonstrated that CGRP levels are elevated in the serum and that blockade of the CGRP pathway results in antinociceptive effects. (Gambeta E, Chichorro J G, Zamponi G W. Trigeminal neuralgia: An overview from pathophysiology to pharmacological treatments. Mol Pain. 2020; 16:1744806920901890) Clinical studies have also demonstrated that CGRP levels are elevated in patients with trigeminal neuralgia compared to healthy patients and decrease significantly after treatment. (Gambeta E. et al.)

CGRP antagonists have shown efficacy for various indications in human clinical trials. See Davis C D, Xu C. Curr Top Med. Chem. 2008 8(16):1468-79; Benemei S, Nicoletti P, Capone J G, Geppetti P. Curr Opin Pharmacol. 2009 9(1):9-14. Epub 2009 Jan. 20; Ho T W, Ferrari M D, Dodick D W, Galet V, Kost J, Fan X, Leibensperger H, Froman S, Assaid C, Lines C, Koppen H, Winner P K. Lancet. 2008 372:2115. Epub 2008 Nov. 25; Ho T W, Mannix L K, Fan X, Assaid C, Furtek C, Jones C J, Lines C R, Rapoport A M; Neurology 2008 70:1304. Epub 2007 Oct. 3.

CGRP receptor antagonists, including small molecule CGRP-receptor antagonists, have been disclosed in PCT publications WO 97/09046, WO 98/09630, WO 98/1128, WO 98/56779, WO 00/18764, WO 00/55154, WO 01/32649, WO 01/49676, WO 01/032648, WO 2004/092166, WO 2004/092168, and WO 2007/120590. See also U.S. Pat. Nos. 6,344,449, 6,313,097, 6,521,609, 6,552,043, US 20030181462, US 20030191068 and WO 03/076432 and related applications. CGRP receptor antagonists have also been disclosed in U.S. Pat. Nos. 8,314,117, 8,759,372, 7,200,862, 8,481,546, and 9,808,457, each of which are incorporated by reference herein in their entirety.

The disclosure provides technical advantages, for example, methods to treat, ameliorate, and/or prevent neuralgia, including trigeminal neuralgia with compounds that inhibit CGRP. Additionally, the methods and compounds provide advantages for pharmaceutical uses, for example, with regard to one or more of their mechanism of action, binding, inhibition efficacy, target selectivity, solubility, safety profiles, or bioavailability.

### SUMMARY OF THE DISCLOSURE

The disclosure generally relates to methods of treating, ameliorating, alleviating, providing for prophylaxis or prevention of, halting the progression of, and/or reducing the risk of neuralgia, including trigeminal neuralgia, comprising administering to a subject in need thereof one or more CGRP receptor antagonists.

In an embodiment, the disclosure describes methods of treating, ameliorating, alleviating, providing for prophylaxis or prevention of, halting the progression of, and/or reducing the risk of neuralgia, including trigeminal neuralgia, comprising administering to a subject in need thereof one or more CGRP receptor antagonists according to one or more of a compound according to Formula (I), Formula (II), and/or Formula (III), or pharmaceutically acceptable salts or optical isomers thereof:

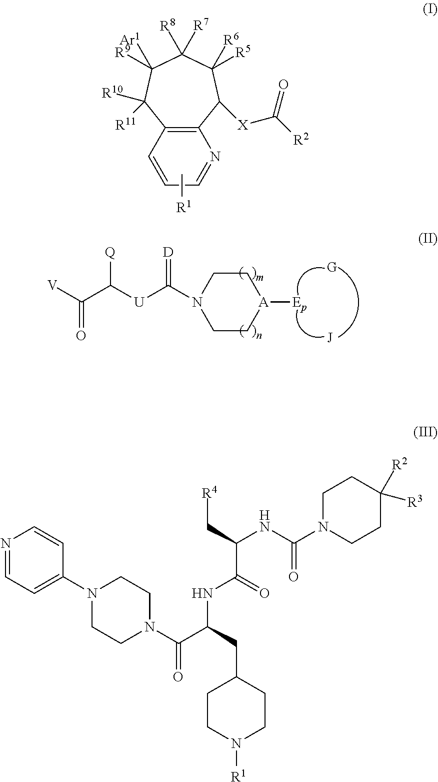

wherein, in Formula (I), R₁ is hydrogen, cyano, alkyl, haloalkyl, alkoxy, amino, alkylamino, dialkylamino, azetidinyl, pyrrolidinyl, or piperidinyl;
R² is piperidinyl substituted with 1 substituent selected from the group consisting of:

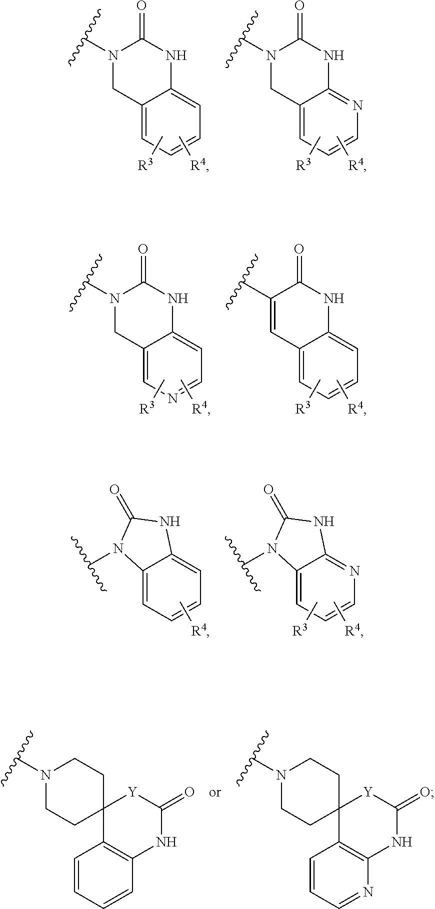

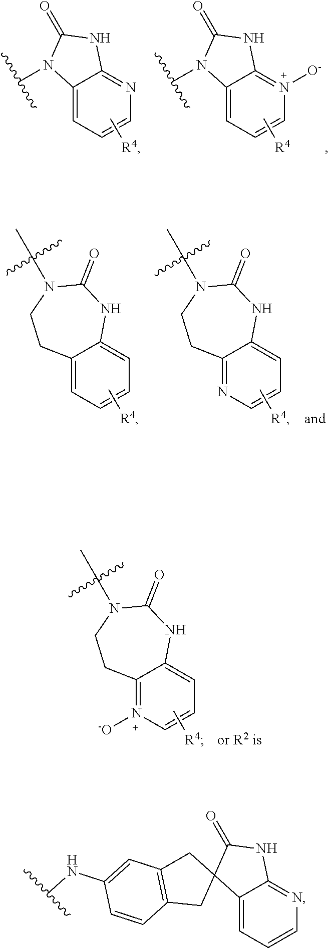

R³ is hydrogen, halo, cyano, alkyl, haloalkyl, alkoxy, or haloalkoxy;
R⁴ is hydrogen, halo, cyano, alkyl, haloalkyl, alkoxy, or haloalkoxy;
R⁵ is hydrogen, hydroxy, alkoxy, haloalkoxy, azido, amino, alkylamino, or dialkylamino;
R⁶ is hydrogen, hydroxy, alkoxy, haloalkoxy, azido, amino, alkylamino, or dialkylamino;
R⁷ is hydrogen, hydroxy, alkoxy, haloalkoxy, azido, amino, alkylamino, or dialkylamino;
R⁸ is hydrogen, hydroxy, alkoxy, haloalkoxy, azido, amino, alkylamino, or dialkylamino;
R⁹ is hydrogen, hydroxy, alkoxy, haloalkoxy, azido, amino, alkylamino, or dialkylamino;
R¹⁰ is hydrogen, hydroxy, alkoxy, haloalkoxy, azido, amino, alkylamino, or dialkylamino;
R¹¹ is hydrogen, hydroxy, alkoxy, haloalkoxy, azido, amino, alkylamino, dialkylamino, alkoxycarbonyl, or benzyloxycarbonyl; or R¹⁰ and R¹¹ taken together is O or N—OH; provided that at least one of R⁵, R⁶, R⁷, R⁸, R⁹, R¹⁰, or R¹¹ is not hydrogen;
Ar¹ is phenyl substituted with 0-3 substituents selected from the group consisting of cyano, halo, alkyl, haloalkyl, alkoxy, haloalkoxy, and alkylSO₂;
X is O, CH₂, or NH; and
Y is a bond, O, CH₂, or NH;
   wherein, in Formula (II), V is —N(R¹)(R²) or OR⁴;
R⁴ is H, C₁₋₆alkyl, C₁₋₄haloalkyl or (C₁₋₄alkylene)₀₋₁R^{4′}
R^{4′} is C₃₋₇cycloalkyl, phenyl, adamantyl, quinucidyl, azabicyclo[2.2.1]heptyl, azetidinyl, tetrahydrofuranyl, furanyl, dioxolanyl, thienyl, tetrahydrothienyl, pyrrolyl, pyrrolinyl, pyrrolidinyl, imidazolyl, imidazolinyl, imidazolidinyl, pyrazolyl, pyrazolinyl, pyrazolidinyl, oxazolyl, isoxazolyl, thiazolyl, isothiazolyl, oxadiazolyl, thiadiazolyl, triazolyl, pyranyl, pyridyl, pyrimidinyl, pyrazinyl, pyridazinyl, triazinyl, piperidinyl, piperazinyl, morpholino, thiomorpholino or dioxolanyl; and
R^{4′} is optionally substituted with 1 or 2 of the same or different substituents selected from the group consisting of halo, cyano, C₁₋₄alkyl, C₁₋₄haloalkyl, C₁₋₄alkoxy, hydroxy, amino, C₃₋₇cycloalkyl, C₁₋₃alkylamino, C₁₋₃dialkylamino, (C₁₋₃alkyl)₀₋₂ureido, phenyl and benzyl; and
R^{4′} optionally contains 1 or 2 carbonyls wherein the carbon atom of said carbonyl is a member of the ring structure of R^{4′};
R¹ and R² are each independently L¹, wherein L¹ is selected from the group consisting of H, C₁₋₆alkyl, C₂₋₆alkenyl, C₂₋₆alkynyl, —C₁₋₆alkylene-amino (C₁₋₃alkyl)₂, C₃₋₇ cycloalkyl, phenyl, azetidinyl, adamantyl, tetrahydrofuranyl, furanyl, dioxolanyl, thienyl, tetrahydrothienyl, pyrrolyl, pyrrolinyl, pyrrolidinyl, imidazolyl, imidazolinyl, imidazolidinyl, pyrazolyl, pyrazolinyl, pyrazolidinyl, oxazolyl, isoxazolyl, thiazolyl, isothiazolyl, oxadiazolyl, thiadiazolyl, triazolyl, triazolyl, pyranyl, pyridyl, pyrimidinyl, pyrazinyl, pyridazinyl, triazinyl, piperidinyl, piperazinyl, morpholino, thiomorpholino and dioxolmnyl; and
R¹ and R² are each optionally and independently substituted with 1 or 2 of the same or different substituents selected from the group consisting of halo, cyano, C₁₋₄alkyl, C₁₋₄haloalkyl, C₁₋₄alkoxy, hydroxy, amino, C₃₋₇cycloalkyl, C₁₋₃alkylamino, C₁₋₄dialkylamino, (C₁₋₃alkyl)₀₋₂ureido, phenyl and benzyl;
R¹ and R² optionally and independently contain 1 or 2 carbonyls wherein the carbon atom of said carbonyl is a member of the heterocycles comprising R¹ and R²
wherein L¹ is optionally and independently interrupted from the nitrogen to which it is attached by L², wherein L² is independently C₁₋₃alkylene or C₁₋₃alkylidene; or
R¹ and R² together with the nitrogen to which they are attached form X,
wherein X is azetidinyl, pyrrolyl, pyrrolinyl, pyrrolidinyl, imidazolizyl, imidazolidinyl, pyrazolinyl, pyrazolidinyl, azepinyl, diazepinyl, piperazinyl, piperidinyl, morpholino or thiomorpholino;
wherein X is optionally substituted with Y, wherein Y is dioxolanyl, C₁₋₉alkyl, C₂₋₂ alkenyl, C₂₋₉alkynyl, C₁₋₄alkylamino, C₁₋₄dialkylamino, C₁₋₄alkoxy, C₃₋₇cycloalkyl, phenyl, azetidinyl, furanyl, thienyl, pyrrolyl, pyrrolinyl, pyrrolidinyl, pyrrolidinonyl, imidazolyl, imidazolinyl, imidazolidinyl, imidazolidinonyl, pyrazolyl, pyrazolinyl, pyrazolidinyl, azepinyl, diazepinyl, pyridyl, pyrimidinyl, dihaydrobenzimidazolonyl, piperazinyl, piperidinyl, morpholino, benzothiazolyl, benzisothiazolyl or thiomorpholino;
and wherein X and Y are optionally interrupted with Z, wherein Z is-NHC(O)O—, —NHC(O)NH—, NC(Q)NH₂, —NH—, —C₁₋₃alkylene-, C₁₋₃alkenylene-NHC(O)O—C₁₋₃ alkylene-; and
optionally and independently substituted with 1 or 2 of the same or different substituents selected from the group consisting of C₁₋₄alkyl, amino, C₁₋₃alkylamino, —C₁₋₃alkylene-amiao(C₁₋₃alkyl)₂, (C₁₋₃alkyl)₀₋₂ureido, phenyl and benzyl;
X and Y optionally and independently contain 1 or 2 carbonyls wherein the carbon atom of said carbonyl is a member of the heterocycles comprising X and Y;
provided that if X is substituted with Y, and if X and Y are not interrupted with Z, then X and Y optionally share one carbon atom and together form a spirocyclic moiety;
Q is Q′ or Q″;
wherein
Q′ is (S^{y})ₛR³; and
Q″ is NH(S^{y})ₛR³, NHC(O)(S^{y})ₛR³, NHC(O)O(S^{y})ₛR³, NHC(O)NH(S^{y}) sR³, O(S^{y})ₛR³, (S^{y})₃NHR³, (S^{y})ₛNHC(O)R³, (S^{y})ₛNHC(O)OR³, (S^{y})ₛNHC(O)NHR³; or (S^{y})ₛOR³;
wherein S^{y} is C₁₋₃alkylene or C₁₋₃alkylidene and a is 0 or 1;
U is CH₂ or NH;
provided that if Q is Q″, then U is CH₂;
R³ is R^{3a} or R^{3b} wherein R^{3a} is
   (i) a heterocycle having two fused rings with 5 to 7 members in each of said rings, said heterocycle containing one to five of the same or different beteroatoms selected from the group consisting of O, N and S, and said heterocycle optionally containing 1 or 2 carbonyls wherein the carbon atom of said carbonyl is a member of said fused rings;
   (ii) a 4 to 6 membered heterocycle containing one to three of the same or different heteroatoms selected from the group consisting of O, N and S, optionally containing 1 to 2 carbonyls, wherein the carbon atom of said carbonyl is a member of said 4 to 6 membered heterocycle;
   (iii) C₃₋₇cycloalkyl;
   (iv) carbazolyl, fluorenyl, phenyl, —O-phenyl, —O—C₁₋₄alklylene-phenyl, or napthyl; or
   (v) C₁₋₈alkyl, C₂₋₇-alkenyl, —C(O)R^{3′}, CHC(O)O—R^{3′}, CH(CH₃)C(O)O—R^{3′}, —C(O)O—R^{3′} or C₂₋₇alkynyl; and
   wherein R^{3a} is optionally substituted with 1 to 3 of the same or different substituents selected from the group consisting of benzyl, phenyl, —O-phenyl, —O—C₁₋₃ alkylenephenyl, —C₁₋₃alkylene-O—C(O)-phenyl, cyano, amino, nitro, halo, C₁₋₆alkyl, C₁₋₃mono-bi-tri-haloalkyl, C₁₋₃mono-bi-tri-haloalkyloxy, (C₁₋₃alkyl)₁₋₂amine, —OR^{3′}, —C(O)R^{3′}, —C(O)O—R^{3′}, —O—C(O)R^{3′}, —N(R^{3′})₂, —C(O)N(R^{3′})₂, —N(R^{3′})C(O)(R^{3′})₂, —N(R^{3′})C(O)N(R^{3′})₂, —N(R^{3′})C(O)OR^{3′}, —O—C(O)N(R^{3′})₂, —N(R^{3′})SO₂R^{3′}, —SO₂N(R^{3′})₂ and —SO₂R^{3′}; R^{3′} is H or —C₁₋₆alkyl;
   provided that if R^{3a} is, —C(O)R^{3′}, CHC(O)O—R^{3′}, CH(CH₃)C(O)O—R^{3′} or —C(O)O—R^{3′}, then said —C(O)R^{3′}, CHC(O)O—R^{3′}, CH(CH₃)C(O)O—R^{3′} or —C(O)O—R^{3′} are unsubstituted;
   R^{3b} is R^{3a} but is not phenyl, 1-naphthyl, 2-naphthyl, 1,2,3,4-tetrahydro-1-naphthyl, 1H-indol-3-yl, 1-methyl-1H-indol-3-yl, 1-formyl-1H-indol-3-yl, 1-(1,1-dirnethylethoxycarbonyl)-1H-indol-3-yl, 4-imidazolyl, 1-methyl-4-imidazolyl, 2-thienyl, 3-thienyl, thiazolyl, 1H-indazol-3-yl, 1-methyl-1H-indazol-3-yl, benzo[b]fur-3-yl, benzo[b]thien-3-yl, pyridinyl, quinolinyl or isoquinolinyl; optionally substituted in the carbon skeleton with mono-, di- or trisubstituted by fluorine, chlorine or bromine atoms or by branched or unbranched alkyl groups, C₃₋₈-cycloalkyl groups, phenylalkyl groups, alkenyl, alkoxy, phenyl, phenylalkoxy, trifluoromethyl, alkoxycoarbonylalkyl, carboxyalkyl, alkoxycarbonyl, carboxy, dialkylaminoalkyl, dialkylaminoalkoxy, hydroxy, nitro, amino, acetylamino, propionylamino, benzoyl, benzoylamino, benzoylmethylamino, methylsulphonyloxy, aminocarbonyl, alkylaminocarbonyl, dialkylaminocarbonyl, alkanoyl, cyano, tetrazolyl, phenyl, pyridinyl, thiazolyl, furyl, trifluoromethoxy, trifluoromethylthio, trifluoromethylsulphinyl- or trifluoromethylsulphonyl groups;
   wherein said substituents may be the same or different and the above-mentioned benzoyl, benzoylaznino- and benzoylmnethylamino groups may in turn additionally be substituted in the phenyl moiety by a fluorine, chlorine or bromine atom, or by an alkyl, trifluoromethyl, amino or acetylamino group;
D is O, NCN or NSO₂C₁₋₃alkyl;
A is CH;
m and n are each 1;
E is N, CE or C;
p is 1;
G, J and E together form A^{x};
   A^{x} is a fused heterocycle having two fused rings with 5 to 7 members in each of said rings, said heterocycle containing one to four of the same or different heteroatoms selected from the group consisting of O, N and S; and
optionally containing 1 or 2 carbonyls wherein the carbon atom of said
carbonyl is a member of said fused heterocycle;
and further provided that
if Q is Q″, then R³ is R^{3a}; and
if Q is Q′, then
R³ is R^{3b}.
   and wherein, in Formula (III), R¹ is H or Q-(C₁-C₆)alkyl;
where Q is a bond, C(O) or C(O)O and where the (C₁-C₆)alkyl can be optionally substituted by N(C₁-C₃alkyl)₂ or CO₂H;
R² is H or forms a spirocyclic heterocyclic ring with R³;
R³ forms a spirocyclic heterocyclic ring with R² or is a heterocyclic ring if R² is H; and
R⁴ is an optionally substituted aryl group which may be monocyclic or fused to a further ring.

In another embodiment, the disclosure describes methods of treating, ameliorating, alleviating, providing for prophylaxis or prevention of, halting the progression of, and/or reducing the risk of neuralgia, including trigeminal neuralgia, comprising administering to a subject in need thereof a pharmaceutical composition comprising one or more CGRP receptor antagonists according to one or more of a compound according to Formula (I), Formula (II), and/or Formula (III).

In another embodiment, the disclosure describes methods of treating, ameliorating, alleviating, providing for prophylaxis or prevention of, halting the progression of, and/or reducing the risk of trigeminal neuralgia, including trigeminal neuralgia, comprising administering to a subject in need thereof a pharmaceutical composition comprising one or more CGRP receptor antagonists according to one or more of a compound according to Formula (I), Formula (II), and/or Formula (III) via an oral administration route, an injection administration route, and/or a topical administration route.

In another embodiment, the disclosure provides for the use of one or more of a compound according to Formula (I), Formula (II), and/or Formula (III) in methods of treating, ameliorating, alleviating, providing for prophylaxis or prevention of, halting the progression of, and/or reducing the risk of trigeminal neuralgia.

In another embodiment, the disclosure provides for the use of a compound according to Formula (I), Formula (II), and/or Formula (III) in the manufacture of a medicament for the treatment of trigeminal neuralgia. In some embodiments, the treatment includes treating, ameliorating, alleviating, providing for prophylaxis or prevention of, halting the progression of, and/or reducing the risk of trigeminal neuralgia.

These and further embodiments will become apparent to a person of skill in the art based upon the disclosure herein.

### DETAILED DESCRIPTION OF THE DISCLOSURE

The disclosure is based upon the surprising discovery that CGRP antagonists of formulas (I), (II), and/or (III) are effective in methods to treat, ameliorate and/or prevent trigeminal neuralgia, as compounds or in pharmaceutical compositions that inhibit the CGRP receptor. Inhibition of the CGRP receptor with one or more CGRP receptor antagonists may prevent symptoms or disease associated with overexpression of CGRP, including trigeminal neuralgia.

### CGRP Receptor Antagonists

CGRP Receptor Antagonists useful for the methods of the present invention may be small molecule (molecular weight<about 2 kDa, or <about 1 kDa) or a larger construct (molecular weight>about 2 kDa, or >1 kDa) such as a peptide, biologic, antibody, etc. In particular, the present disclosure is drawn to methods of treating trigeminal neuralgia comprising administering to a mammalian or human subject one or more CGRP receptor antagonists. Such CGRP receptor antagonists may be described by various generic or specific chemical formulas. In the following exemplary chemical formulas, the general structure broadly describes a number of chemical structures in the alternative and it can be appreciated that each alternative structure is contemplated.

### Formula I

In some embodiments, a method of treating, ameliorating, alleviating, providing for prophylaxis or prevention of, halting the progression of, and/or reducing the risk of trigeminal neuralgia, may comprise administering to a subject in need thereof a CGRP receptor antagonist according to Formula (I):

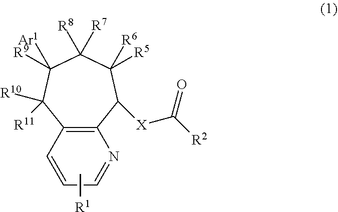

where:
R¹ is hydrogen, cyano, alkyl, haloalkyl, alkoxy, amino, alkylamino, dialkylamino, azetidinyl, pyrrolidinyl, or piperidinyl;
R² is piperidinyl substituted with 1 substituent selected from the group consisting of:

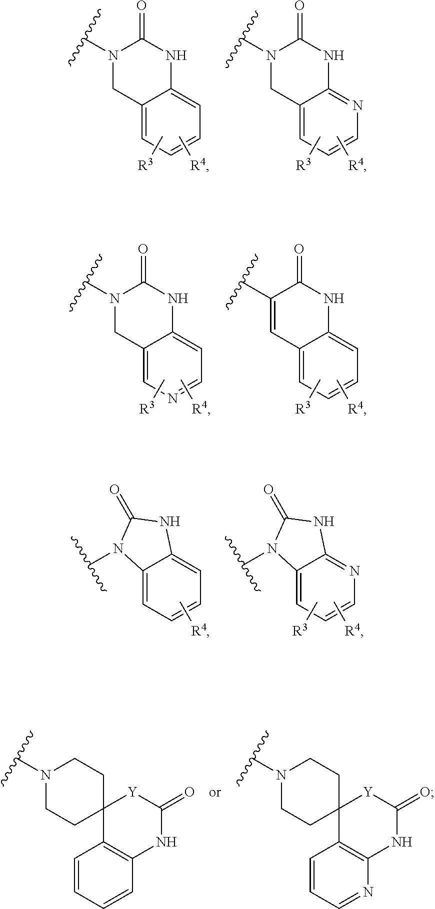

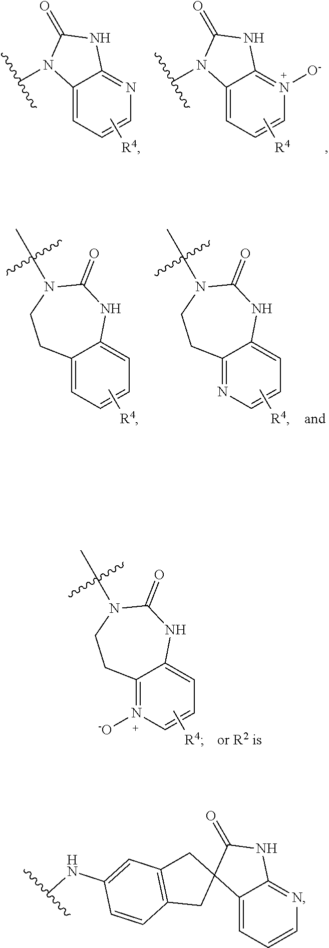

R³ is hydrogen, halo, cyano, alkyl, haloalkyl, alkoxy, or haloalkoxy;
R⁴ is hydrogen, halo, cyano, alkyl, haloalkyl, alkoxy, or haloalkoxy;
R⁵ is hydrogen, hydroxy, alkoxy, haloalkoxy, azido, amino, alkylamino, or dialkylamino;
R⁶ is hydrogen, hydroxy, alkoxy, haloalkoxy, azido, amino, alkylamino, or dialkylamino;
R⁷ is hydrogen, hydroxy, alkoxy, haloalkoxy, azido, amino, alkylamino, or dialkylamino;
R⁸ is hydrogen, hydroxy, alkoxy, haloalkoxy, azido, amino, alkylamino, or dialkylamino;
R⁹ is hydrogen, hydroxy, alkoxy, haloalkoxy, azido, amino, alkylamino, or dialkylamino;
R¹⁰ is hydrogen, hydroxy, alkoxy, haloalkoxy, azido, amino, alkylamino, or dialkylamino;
R¹¹ is hydrogen, hydroxy, alkoxy, haloalkoxy, azido, amino, alkylamino, dialkylamino, alkoxycarbonyl, or benzyloxycarbonyl; or R¹⁰ and R¹¹ taken together is O or N—OH; provided that at least one of R⁵, R⁶, R⁷, R⁸, R⁹, R¹⁰, or R¹¹ is not hydrogen;
Ar¹ is phenyl substituted with 0-3 substituents selected from the group consisting of cyano, halo, alkyl, haloalkyl, alkoxy, haloalkoxy, and alkylSO₂;

### X is O, CH₂, or NH; and

Y is a bond, O, CH₂, or NH;
or a pharmaceutically acceptable salt or optical isomer thereof.
In some embodiments, the CGRP receptor antagonists described by general Formula I may be selected from any of the following specific formulas:
(6R,9R)-6-(2,3-Difluorophenyl)-6-hydroxy-5-oxo-6,7,8,9-tetrahydro-5H-cyclohepta[b]pyridin-9-yl 4-(2-oxo-2,3-dihydro-1H-imidazo[4,5-b]pyridin-1-yl)piperidine-1-carboxylate;
(9R)-6-(2,3-difluorophenyl)-5-oxo-6,7,8,9-tetrahydro-5H-cyclohepta[b]pyridin-9-yl 4-(2-oxo-2,3-dihydro-1H-imidazo[4,5-b]pyridin-1-yl)piperidine-1-carboxylate;
(5S,6R,9R)-6-(2,3-difluorophenyl)-5,6-dihydroxy-6,7,8,9-tetrahydro-5H-cyclohepta[b]pyridin-9-yl 4-(2-oxo-2,3-dihydro-1H-imidazo[4,5-b]pyridin-1-yl)piperidine-1-carboxylate;
(5S,6S,9R)-6-(2,3-difluorophenyl)-5-hydroxy-6,7,8,9-tetrahydro-5H-cyclohepta[b]pyridin-9-yl 4-(2-oxo-2,3-dihydro-1H-imidazo[4,5-b]pyridin-1-yl)piperidine-1-carboxylate;
(5S,6S,9R)-6-(2,3-difluorophenyl)-5-hydroxy-6,7,8,9-tetrahydro-5H-cyclohepta[b]pyridin-9-yl 4-(2-oxo-2,3-dihydro-1H-imidazo[4,5-b]pyridin-1-yl)piperidine-1-carboxylate;
(5S,6R,9R)-6-(2,3-difluorophenyl)-5-hydroxy-6,7,8,9-tetrahydro-5H-cyclohepta[b]pyridin-9-yl 4-(2-oxo-2,3-dihydro-1H-imidazo[4,5-b]pyridin-1-yl)piperidine-1-carboxylate;
(5S,6S,9R)-5-azido-6-(2,3-difluorophenyl)-6,7,8,9-tetrahydro-5H-cyclohepta[b]pyridin-9-yl 4-(2-oxo-2,3-dihydro-1H-imidazo[4,5-b]pyridin-1-yl)piperidine-1-carboxylate;
(5S,6S,9R)-5-amino-6-(2,3-difluorophenyl)-6,7,8,9-tetrahydro-5H-cyclohepta[b]pyridin-9-yl 4-(2-oxo-2,3-dihydro-1H-imidazo[4,5-b]pyridin-1-yl)piperidine-1-carboxylate;
(6S,9R)-6-(2,3-difluorophenyl)-6-hydroxy-6,7,8,9-tetrahydro-5H-cyclohepta[b]pyridin-9-yl 4-(2-oxo-2,3-dihydro-1H-imidazo[4,5-b]pyridin-1-yl)piperidine-1-carboxylate;
(5S,6S,9R)-5-amino-6-(3,5-difluorophenyl)-6,7,8,9-tetrahydro-5H-cyclohepta[b]pyridin-9-yl 4-(2-oxo-2,3-dihydro-1H-imidazo[4,5-b]pyridin-1-yl)piperidine-1-carboxylate;
(5S,6S,9R)-6-(3,5-difluorophenyl)-5-hydroxy-6,7,8,9-tetrahydro-5H-cyclohepta[b]pyridin-9-yl 4-(2-oxo-2,3-dihydro-1H-imidazo[4,5-b]pyridin-1-yl)piperidine-1-carboxylate;
(6S,8R,9S)-6-(2,3-difluorophenyl)-8-hydroxy-6,7,8,9-tetrahydro-5H-cyclohepta[b]pyridin-9-yl 4-(2-oxo-2,3-dihydro-1H-imidazo[4,5-b]pyridin-1-yl)piperidine-1-carboxylate;
(5S,6S,9R)-6-(2,3-difluorophenyl)-5-(methylamino)-6,7,8,9-tetrahydro-5-H-cyclohepta[b]pyridin-9-yl 4-(2-oxo-2,3-dihydro-1H-imidazo[4,5-b]pyridin-1-yl)piperidine-1-carboxylate;
(5S,6S,9R)-6-(2,3-difluorophenyl)-5-(dimethylamino)-6,7,8,9-tetrahydro-5H-cyclohepta[b]pyridin-9-yl 4-(2-oxo-2,3-dihydro-1H-imidazo[4,5-b]pyridin-1-yl)piperidine-1-carboxylate;
(5S,9R,Z)-6-(2,3-difluorophenyl)-5-(hydroxyimino)-6,7,8,9-tetrahydro-5-H-cyclohepta[b]pyridin-9-yl 4-(2-oxo-2,3-dihydro-1H-imidazo[4,5-b]pyridin-1-yl)piperidine-1-carboxylate;
(6S,9R,E)-6-(2,3-difluorophenyl)-5-(hydroxyimino)-6,7,8,9-tetrahydro-5-H-cyclohepta[b]pyridin-9-yl 4-(2-oxo-2,3-dihydro-1H-imidazo[4,5-b]pyridin-1-yl)piperidine-1-carboxylate;
(5S,6S,9R)-5-amino-6-(2,3-difluorophenyl)-6,7,8,9-tetrahydro-5H-cyclohepta[b]pyridin-9-yl 2′-oxo-1,1′,2′,3-tetrahydrospiro[indene-2,3′-pyrrolo[2,3-b]pyridine]-5-yl-carbamate;
tert-butyl(5S,6S,9R)-9-amino-6-(2,3-difluorophenyl)-6,7,8,9-tet-rahydro-5H-cyclohepta[b]pyridin-5-ylcarbamate; tert-butyl (5S,6S,9S)-6-(2,3-difluorophenyl)-9-(2-oxo-2-(4-(2-oxo-2,3-dihydro-1H-imi-dazo[4,5-b]pyridin-1-yl)piperidin-1-yl)ethyl)-6,7,8,9-tetrahydro-5H-cycloh-epta[b]pyridin-5-yl-carbamate;
tert-butyl (5S,6S,9R)-6-(2,3-difluorophenyl)-9-(2-oxo-2-(4-(2-oxo-2,3-dihydro-1H-imi-dazo[4,5-b]pyridin-1-yl)piperidin-1-yl)ethyl)-6,7,8,9-tetrahydro-5H-cycloh-epta[b]pyridin-5-ylcarbamate; and
1-(1-(2-((5S,6S,9R)-5-amino-6-(2,3-difluorophenyl)-6,7,8,9-tetrahydro-5H-cyclohepta[b]pyridin-9-yl)acetyl)piperidin-4-yl)-1H-imidazo[4,5-b]pyridin-2(3H)-one; or a pharmaceutically acceptable salt thereof.

In some embodiments, the CGRP receptor antagonist may be Rimegepant, which has the formula (5S,6S,9R)-5-amino-6-(2,3-difluorophenyl)-6,7,8,9-tetrahydro-5H-cyclohepta[b]pyridine-9-yl-4-(2-oxo-2,3-dihydro-1H-imidazo[4,5-b]pyridine-1-yl)piperidine-1-carboxylate, and which has the following structure

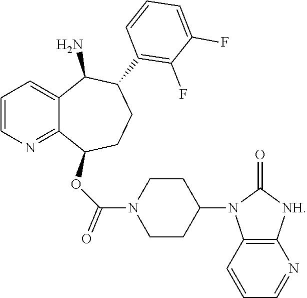 [SMILES: N[C@@H]1c2cccnc2[C@H](OC(=O)N2CCC(n3c(=O)[nH]c4ncccc43)CC2)CC[C@H]1c1cccc(F)c1F]

or a pharmaceutically acceptable salt thereof, including the hemisulfate salt and the hemisulfate sesquihydrate.

### Formula II

In some embodiments, a method of treating, ameliorating, alleviating, providing for prophylaxis or prevention of, halting the progression of, and/or reducing the risk of trigeminal neuralgia, may comprise administering to a subject in need thereof a CGRP receptor antagonist according to Formula (II):

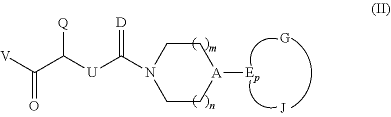

or a pharmaceutically acceptable salt or optical isomer thereof,
wherein
V is —N(R¹)(R²) or OR⁴;
R⁴ is H, C₁₋₆alkyl, C₁₋₄haloalkyl or (C₁₋₄alkylene)₀₋₁R^{4′}
R^{4′} is C₃₋₇cycloalkyl, phenyl, adamantyl, quinucidyl, azabicyclo[2.2.1]heptyl, azetidinyl, tetrahydrofuranyl, furanyl, dioxolanyl, thienyl, tetrahydrothienyl, pyrrolyl, pyrrolinyl, pyrrolidinyl, imidazolyl, imidazolinyl, imidazolidinyl, pyrazolyl, pyrazolinyl, pyrazolidinyl, oxazolyl, isoxazolyl, thiazolyl, isothiazolyl, oxadiazolyl, thiadiazolyl, triazolyl, pyranyl, pyridyl, pyrimidinyl, pyrazinyl, pyridazinyl, triazinyl, piperidinyl, piperazinyl, morpholino, thiomorpholino or dioxolanyl; and
R^{4′} is optionally substituted with 1 or 2 of the same or different substituents selected from the group consisting of halo, cyano, C₁₋₄alkyl, C₁₋₄haloalkyl, C₁₋₄alkoxy, hydroxy, amino, C₃₋₇cycloalkyl, C₁₋₃alkylamino, C₁₋₃dialkylamino, (C₁₋₃alkyl)₀₋₂ureido, phenyl and benzyl; and
R^{4′} optionally contains 1 or 2 carbonyls wherein the carbon atom of said carbonyl is a member of the ring structure of R^{4′};
R¹ and R² are each independently L¹, wherein L¹ is selected from the group consisting of H, C₁₋₆alkyl, C₂₋₆alkenyl, C₂₋₆alkynyl, —C₁₋₆alkylene-amino (C₁₋₃alkyl)₂, C₃-7cycloalkyl, phenyl, azetidinyl, adamantyl, tetrahydrofuranyl, furanyl, dioxolanyl, thienyl, tetrahydrothienyl, pyrrolyl, pyrrolinyl, pyrrolidinyl, imidazolyl, imidazolinyl, imidazolidinyl, pyrazolyl, pyrazolinyl, pyrazolidinyl, oxazolyl, isoxazolyl, thiazolyl, isothiazolyl, oxadiazolyl, thiadiazolyl, triazolyl, triazolyl, pyranyl, pyridyl, pyrimidinyl, pyrazinyl, pyridazinyl, triazinyl, piperidinyl, piperazinyl, morpholino, thiomorpholino and dioxolmnyl; and
R¹ and R² are each optionally and independently substituted with 1 or 2 of the same or different substituents selected from the group consisting of halo, cyano, C₁₋₄alkyl, C₁₋₄ haloalkyl, C₁₋₄alkoxy, hydroxy, amino, C₃₋₇cycloalkyl, C₁₋₃alkylamino, C₁₋₄dialkylamino, (C₁₋₃ alkyl)₀₋₂ureido, phenyl and benzyl;
R¹ and R² optionally and independently contain 1 or 2 carbonyls wherein the carbon atom of said carbonyl is a member of the heterocycles comprising R¹ and R²; wherein L¹ is optionally and independently interrupted from the nitrogen to which it is attached by L², wherein L² is independently C₁₋₃alkylene or C₁₋₃alkylidene; or
R¹ and R² together with the nitrogen to which they are attached form X,
wherein X is azetidinyl, pyrrolyl, pyrrolinyl, pyrrolidinyl, imidazolizyl, imidazolidinyl, pyrazolinyl, pyrazolidinyl, azepinyl, diazepinyl, piperazinyl, piperidinyl, morpholino or thiomorpholino;
wherein X is optionally substituted with Y, wherein Y is dioxolanyl, C₁₋₉alkyl, C₂₋₉ alkenyl, C₂₋₉alkynyl, C₁₋₄alkylamino, C₁₋₄dialkylamino, C₁₋₄alkoxy, C₃₋₇cycloalkyl, phenyl, azetidinyl, furanyl, thienyl, pyrrolyl, pyrrolinyl, pyrrolidinyl, pyrrolidinonyl, imidazolyl, imidazolinyl, imidazolidinyl, imidazolidinonyl, pyrazolyl, pyrazolinyl, pyrazolidinyl, azepinyl, diazepinyl, pyridyl, pyrimidinyl, dihaydrobenzimidazolonyl, piperazinyl, piperidinyl, morpholino, benzothiazolyl, benzisothiazolyl or thiomorpholino;
and wherein X and Y are optionally interrupted with Z, wherein Z is-NHC(O)O—, —NHC(O)NH—, NC(Q)NH₂, —NH—, —C₁₋₃alkylene-, C₁₋₃alkenylene-NHC(O)O—C₁-3alkylene-; and
optionally and independently substituted with 1 or 2 of the same or different substituents selected from the group consisting of C₁₋₄alkyl, amino, C₁₋₃alkylamino, —C₁₋₃ alkylene-amiao(C₁₋₃alkyl)₂, (C₁₋₃alkyl)₀₋₂ureido, phenyl and benzyl;
X and Y optionally and independently contain 1 or 2 carbonyls wherein the carbon atom of said carbonyl is a member of the heterocycles comprising X and Y;
provided that if X is substituted with Y, and if X and Y are not interrupted with Z, then X and Y optionally share one carbon atom and together form a spirocyclic moiety;
Q is Q′ or Q″;
wherein
Q′ is (S^{y})ₛR³; and
Q″ is NH(S^{y})ₛR³, NHC(O)(S^{y})ₛR³, NHC(O)O(S^{y})ₛR³, NHC(O)NH(S^{y}) sR³, O(S^{y})ₛR³, (S^{y})₃NHR³, (S^{y})ₛNHC(O)R³, (S^{y})ₛNHC(O)OR³, (S^{y})ₛNHC(O)NHR³; or (S^{y})ₛOR³;
wherein S^{y} is C₁₋₃alkylene or C₁₋₃alkylidene and a is 0 or 1;
U is CH₂ or NH;
provided that if Q is Q″, then U is CH₂;
R³ is R^{3a} or R^{3b} wherein R^{3a} is
(i) a heterocycle having two fused rings with 5 to 7 members in each of said rings, said heterocycle containing one to five of the same or different beteroatoms selected from the group consisting of O, N and S, and said heterocycle optionally containing 1 or 2 carbonyls wherein the carbon atom of said carbonyl is a member of said fused rings;
(ii) a 4 to 6 membered heterocycle containing one to three of the same or different heteroatoms selected from the group consisting of O, N and S, optionally containing 1 to 2 carbonyls, wherein the carbon atom of said carbonyl is a member of said 4 to 6 membered heterocycle;
(iii) C₃₋₇cycloalkyl;
(iv) carbazolyl, fluorenyl, phenyl, —O-phenyl, —O—C₁₋₄alklylene-phenyl, or napthyl; or
(v) C₁₋₈alkyl, C₂₋₇alkenyl, —C(O)R^{3′}, CHC(O)O—R^{3′}, CH(CH₃)C(O)O—R^{3′}, —C(O)O—R^{3′} or C₂₋₇alkynyl; and
wherein R^{3a} is optionally substituted with 1 to 3 of the same or different substituents selected from the group consisting of benzyl, phenyl, —O-phenyl, —O—C₁₋₃ alkylenephenyl, —C₁₋₃alkylene-O—C(O)-phenyl, cyano, amino, nitro, halo, C₁₋₆alkyl, C₁₋₃ mono-bi-tri-haloalkyl, C₁₋₃mono-bi-tri-haloalkyloxy, (C₁₋₃alkyl)₁₋₂amine, —OR^{3′}, —C(O)R^{3′}, —C(O)O—R^{3′}, —O—C(O)R^{3′}, —N(R^{3′})₂, —C(O)N(R^{3′})₂, —N(R^{3′})C(O)(R^{3′})₂, —N(R^{3′})C(O)N(R^{3′})₂, —N(R^{3′})C(O)OR^{3′}, —O—C(O)N(R^{3′})₂, —N(R^{3′})SO₂R^{3′}, —SO₂N(R^{3′})₂ and —SO₂R^{3′}; R^{3′} is H or —C₁₋₆alkyl;
provided that if R^{3a} is, —C(O)R^{3′}, CHC(O)O—R^{3′}, CH(CH₃)C(O)O—R^{3′} or —C(O)O—R^{3′}, then said —C(O)R^{3′}, CHC(O)O—R^{3′}, CH(CH₃)C(O)O—R^{3′} or —C(O)O—R^{3′} are unsubstituted;
R^{3b} is R^{3a} but is not phenyl, 1-naphthyl, 2-naphthyl, 1,2,3,4-tetrahydro-1-naphthyl, 1H-indol-3-yl, 1-methyl-1H-indol-3-yl, 1-formyl-1H-indol-3-yl, 1-(1,1-dirnethylethoxycarbonyl)-1H-indol-3-yl, 4-imidazolyl, 1-methyl-4-imidazolyl, 2-thienyl, 3-thienyl, thiazolyl, 1H-indazol-3-yl, 1-methyl-1H-indazol-3-yl, benzo[b]fur-3-yl, benzo[b]thien-3-yl, pyridinyl, quinolinyl or isoquinolinyl; optionally substituted in the carbon skeleton with mono-, di- or trisubstituted by fluorine, chlorine or bromine atoms or by branched or unbranched alkyl groups, C₃₋₈-cycloalkyl groups, phenylalkyl groups, alkenyl, alkoxy, phenyl, phenylalkoxy, trifluoromethyl, alkoxycoarbonylalkyl, carboxyalkyl, alkoxycarbonyl, carboxy, dialkylaminoalkyl, dialkylaminoalkoxy, hydroxy, nitro, amino, acetylamino, propionylamino, benzoyl, benzoylamino, benzoylmethylamino, methylsulphonyloxy, aminocarbonyl, alkylaminocarbonyl, dialkylaminocarbonyl, alkanoyl, cyano, tetrazolyl, phenyl, pyridinyl, thiazolyl, furyl, trifluoromethoxy, trifluoromethylthio, trifluoromethylsulphinyl- or trifluoromethylsulphonyl groups;
wherein said substituents may be the same or different and the above-mentioned benzoyl, benzoylaznino- and benzoylmnethylamino groups may in turn additionally be substituted in the phenyl moiety by a fluorine, chlorine or bromine atom, or by an alkyl, trifluoromethyl, amino or acetylamino group;
D is O, NCN or NSO₂C₁₋₃alkyl;
A is CH;
m and n are each 1;
E is N, CE or C;
p is 1;
   G, J and E together form A^{x};
A^{x} is a fused heterocycle having two fused rings with 5 to 7 members in each of said rings, said heterocycle containing one to four of the same or different heteroatoms selected from the group consisting of O, N and S; and
optionally containing 1 or 2 carbonyls wherein the carbon atom of said
carbonyl is a member of said fused heterocycle;
and further provided that
if Q is Q″, then R³ is R^{3a}; and
if Q is Q′, then
R³ is R^{3b}.

In some embodiments, the CGRP receptor antagonists described by general Formula II may be selected from any of the following specific formulas:
(±)-3-(1H-Indazol-5-yl)-2-{[4-(2-oxo-1,4-dihydro-2H-quinazolin-3-yl)-piperidine-1-carbonyl]-amino}-propionic acid;
(R)-4-(2-Oxo-1,4-dihydro-2H-quinazolin-3-yl)-piperidine-1-carboxylic acid [2-[1,4′]bipiperidinyl-1′-yl-1-(1H-indazol-5-ylmethyl)-2-oxo-ethyl]-amide;
(±)-4-(2-Oxo-2,3-dihydro-benzoimidazol-1-yl)-piperidine-1-carboxylic acid [2-[1,4′]bipiperidinyl-1′-yl-1-(1H-indol-5-ylmethyl)-2-oxo-ethyl]-am-ide;
(±)-4-(2-Oxo-2,3-dihydro-benzoimidazol-1-yl)-piperidine-1-carboxylic acid [2-[1,4′]bipiperidinyl-1′-yl-1-(1H-indazol-5-ylmethyl)-2-oxo-ethyl-]-amide;
(±)-4-(2-Oxo-1,4-dihydro-2H-quinazolin-3-yl)-piperidine-1-carboxylic acid [2-[1,4′]bipiperidinyl-1′-yl-1-(1H-indol-5-ylmethyl)-2-oxo-ethyl]-amide;
(±)-4-(2-Oxo-1,4-dihydro-2H-quinazolin-3-yl)-piperidine-1-carboxylic acid [2-[1,4′]bipiperidinyl-1′-yl-1-(1H-indazol-5-ylmethyl)-2-oxo-ethyl]-amide;
(±)-4-(2-Oxo-2,3-dihydro-benzoimidazol-1-yl)-piperidine-1-carboxylic acid [1-(1H-indol-5-ylmethyl)-2-(4-isobutyl-piperazin-1-yl)-2-oxo-ethyl]-amide;
(±)-4-(2-Oxo-2,3-dihydro-benzoimidazol-1-yl)-piperidine-1-carboxylic acid [2-(1,4-dioxa-8-aza-spiro[4.5]dec-8-yl)-1-(1H-indol-5-ylmethyl)-2-ox-o-ethyl]-amide;
(±)-4-(2-Oxo-2,3-dihydro-benzoimidazol-1-yl)-piperidine-1-carboxylic acid [1-(1H-indazol-5-ylmthyl)-2-(4-isobutyl-piperazin-1-yl)-2-oxo-ethyl]-amide;
(±)-4-(2-Oxo-2,3-dihydro-benzoimidazol-1-yl)-piperidine-1-carboxylic acid [2-(1,4-dioxa-8-aza-spiro[4.5]dec-8-yl)-1-(1H-indazol-5-ylmethyl)-2-oxo-ethyl]-amide;
(±)-4-(2-Oxo-1,4-dihydro-2H-quinazolin-3-yl)-piperidine-1-carboxylic acid [2-(1,4-dioxa-8-aza-spiro[4.5]dec-8-yl)-1-(1H-indazol-5-ylmethyl)-2-oxo-ethyl]-amide;
(±)-3-(7-Methyl-1H-indazol-5-yl)-2-{[4-(2-oxo-1,4-dihydro-2H-quinazoli-n-3-yl)-piperidine-1-carbonyl]-amino}-propionic acid methyl ester;
(±)-3-(7-Methyl-1H-indazol-5-yl)-2-{[4-(2-oxo-1,4-dihydro-2H-quinazoli-n-3-yl)-piperidine-1-carbonyl]-amino}-propionic acid;
(±)-4-(2-Oxo-1,4-dihydro-2H-quinazolin-3-yl)-piperidine-1-carboxylic acid [2-[1,4′]bipiperidinyl-1′-yl-1-(7-methyl-1H-indazol-5-ylmethyl)-2-ox-o-ethyl]-amide;
(±)-4-(2-Oxo-1,4-dihydro-2H-quinazolin-3-yl)-piperidine-1-carboxylic acid [1-(7-methyl-1H-indazol-5-ylmethyl)-2-oxo-2-piperidin-1-yl-ethyl]-amide;
(±)4-(2-Oxo-1,4-dihydro-2H-quinazolin-3-yl)-piperidine-1-carboxylic acid [1-dimethylcarbamoyl-2-(7-methyl-1H-indazol-5-yl)-ethyl]-amide;
(±)-4-(2-Oxo-1,4-dihydro-2H-quinazolin-3-yl)-piperidine-1-carboxylic acid [1-1-(7-methyl-1H-indazol-5-ylmethyl)-2-(4-methyl-piperazin-1-yl)-2-oxo-ethyl]-amide;
(±)-4-(2-Oxo-1,4-dihydro-2H-quinazolin-3-yl)-piperidine-1-carboxylic acid [1-1-(7-methyl-1H-indazol-5-ylmethyl)-2-oxo-2-pyrrolidin-1-yl-ethyl]-amide;
(±)-4-(2-Oxo-1,4-dihydro-2H-quinazolin-3-yl)-piperidine-1-carboxylic acid [1-(7-methyl-1H-indazol-5-ylmethyl)-2-oxo-2-(4-pyridin-4-yl-piperazin-1-yl)-ethyl]-amide;
(±)-4-(2-Oxo-1,4-dihydro-2H-quinazolin-3-yl)-piperidine-1-carboxylic acid [1-(7-methyl-1H-indazol-5-ylmethyl)-2-oxo-2-(4-pyridin-2-yl-piperazin-1-yl)-ethyl]-amide;
4-(2-Oxo-1,4-dihydro-2H-quinazolin-3-yl)-piperidine-1-carboxylic acid {1-(1-1H-indazol-5-ylmethyl)-2-oxo-2-[4-(2-oxo-1,4-dihydro-2H-quinazolin-3-yl)-piperidine-1-yl]-ethyl}-amide;
4-(3-(1H-Indazol-5-yl)-2-{[4-(2-oxo-1,4-dihydro-2H-quinazolin-3-yl)-piperidine-1-carbonyl]-amino}-propionyl)-piperazine-1-carboxylic acid benzyl ester;
4-(2-Oxo-1,4-dihydro-2H-quinazolin-3-yl)-piperidine-1-carboxylic acid [1-(1H-indazol-5-ylmethyl)-2-oxo-2-piperazin-1-yl-ethyl]-amide;
4-(2-Oxo-1,4-dihydro-2H-quinazolin-3-yl)-piperidine-1-carboxylic acid {11-(1H-indazol-5-ylmethyl)-2-[4-(2-methyl-butyl)-piperazin-1-yl]-2-oxo-et-hyl}-amide;

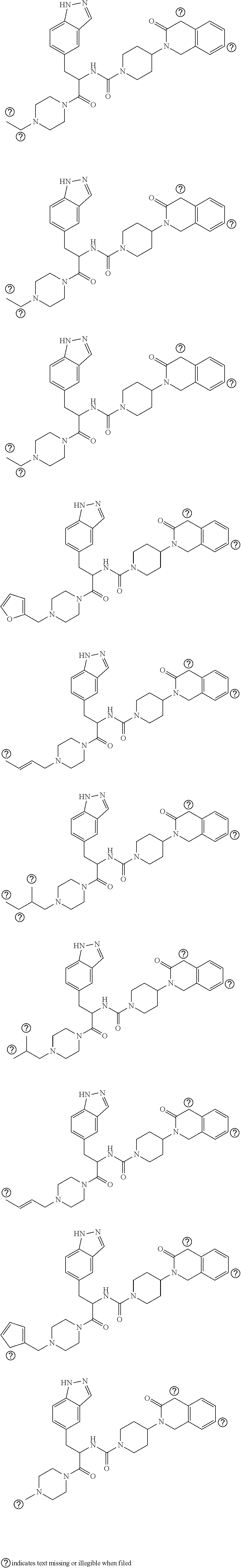

3-(7-Methyl-1H-indazol-5-yl)-2-{[4-(2-oxo-1,4-dihydro-2H-quinazolin-3-yl)-piperidine-1-carbonyl]-amino}-propionic acid cyclohexyl ester;
3-(7-Methyl-1H-indazol-5-yl)-2-{[4-(2-oxo-1,4-dihydro-2H-quinazolin-3-yl)-piperidine-1-carbonyl]-amino}-propionic acid 1-benzyl-piperidin-4-yl ester;
3-(7-Methyl-1H-indazol-5-yl)-2-{[4-(2-oxo-1,4-dihydro-2H-quinazoli-n-3-yl)-piperidine-1-carbonyl]-amino}-propionic acid 1-methyl-piperidin-4-yl ester,
3-(7-Methyl-1H-indazol-5-yl)-2-{[4-(2-oxo-1,4-dihydro-2H-quinazolin-3-yl)-piperidine-1-carbonyl]-amino}-propionic acid 4-phenyl-cyclohexyl ester;
3-(7-Methyl-1H-indazol-5-yl)-2-{[4-(2-oxo-1,4-dihydro-2H-quinazolin-3-yl)-piperidine-1-carbonyl]-amino}-propionic acid (R)-1-pyridin-4-yl-etyl ester;
3-(7-Methyl-1H-indazol-5-yl)-2-{[4-(2-oxo-1,4-dihydro-2H-quinazoli-n-3-yl)-piperidine-1-carbonyl]-amino}-propionic acid (S)-1-pyridin-4-yl-ethyl ester;
(±)-3-(7-Chloro-1H-indazol-5-yl)-2-{[4-(2-oxo-1,4-dihydro-2H-quinazoli-n-3-yl)-piperidine-1-carbonyl]-amino}-propionic acid methyl ester;
(±)-4-(2-Oxo-1,4-dihydro-2H-quinazolin-3-yl)-pipeine-1-carboxylic acid [2-[1,4′]bipiperidinyl-1′-yl-1-(7-chloro-1H-indazol-5-ylmethyl)-2-oxo-ethyl]-amide;
(±)-3-(7-Ethyl-1H-indazol-5-yl)-2-{[4-(2-oxo-1,4-dihydro-2H-quinazolin-3-yl)piperidine-1-carbonyl]-amino}-propionic acid methyl ester
(±)-4-(2-Oxo-1,4-dihydro-2H-quinazolin-3-yl)-piperidine-1-carboxylic acid [2-[1,4′]bipiperidinyl-1′-yl-1-(7-ethyl-1H-indazol-5-ylmethyl)-2oxo-ethyl]-amide;
(R)-4-(2-Oxo-1,4-dihydro-2H-quinazolin-3-yl)-piperidine-1-carboxylic acid [2-[1,4′]bipiperidinyl-1′-yl-1-(2-methyl-1H-benzoimidazol-5-ylmethyl)-2-o-xo-ethyl]-amide;
(R)-4(2-Oxo-1,4-dihydro-2H-quinazolin-3-yl)-piperidine-1-carboxylic acid [2-[1,4′]bipiperidinyl-1′-yl-2-oxo-1-(2-oxo-2,3-dihydro-benzooxazol-6-ylm-ethyl)-ethyl]-amide;
(R)-3-(1H-Benzotriazol-5-yl)-2-{[4-(2-oxo-1,4-dihydro-2H-quinazolin-3-yl)-piperidine-1-carbonyl]-amino}-propionic acid methyl ester;
(R)-4-(2-Oxo-1,4-dihydro-2H-quinazolin-3-yl)-piperidine-1-carboxylic acid [1-(1H-benzotriazol-5-ylmethyl)-2-[1,4′]bipiperidinyl-1′-yl-2-oxo-ethyl]-amide;
(R)-4-(2-Oxo-1,4-dihydro-2H-quinazolin-3-yl)-piperidine-1-carboxylic acid [2[1,4′]bipiperidinyl-1′-yl-2-oxo-1-(2-oxo-2,3-dihydro-1H-indol-5-y-Imethyl)-ethyl]-amide;
(±)-4(2-Oxo-1,4-dihydro-2H-quinazolin-3-yl)-piperidine-1-carboxylic acid [1-(4-benzyloxy-2-oxo-2H pyridin-1-ylmethyl)-2-[1,4′]bipiperidinyl-1′-yl-2-oxo-ethyl]-amide;
(±)-4-(2-Oxo-1,4-dihydro-2H-quinazolin-3-yl)-piperidine-1-carboxylic acid [2-[1,4′bipiperidnyl-1′-yl-1-(4-hydroxy-2-oxo-2H-pyridin-1-ylmethyl)-2-oxo-ethyl]-amide;
(±)-4-(2-Oxo-1,4-dihydro-2H-quinazolin-3-yl)-piperidine-1-carboxylic acid [2-[1,4′bipiperidinyl-1′-yl-1-(4-hydroxy-piperidin-1-ylmethyl)-2-oxo-ethy]-amide;
(±)-4-(2-Oxo-1,4-dihydro-2H-quinazolin-3-yl)-piperidine-1-carboxylic acid [2-[1,4′]bipiperidinyl-1′-yl-2-oxo-1-(1,4,6,7-tetrahydro-pyrazolo[4,-3-c]pyridin-5-ylmethyl)-ethyl]-amide;
(±)-3-(7,7-Dimethyl-1,4,6,7-tetrahydro-pyrazolo[4,3-c]pyridin-5-yl)-2-{[4-(2-oxo-1,4-dihydro-2H-quinazolin-3-yl)-piperidine-1-carbonyl]-amino}-propionic acid methyl ester;
(±)-4-(2-Oxo-1,4-dihydro-2H-quinazolin-3-yl)-piperidine-1-carboxylic acid [2[1,4′bipiperidinyl-1′-yl-1-(7,7-dimethyl-1,4,6,7-tetrahydro-pyrazolo[4,3-c]pyridin-5-ylmethyl)-2-oxo-ethyl]-amide;
(±)-4(2Oxo-1,4-dihydro-2H-quinazolin-3-yl)-piperidine-1-carboxylic acid [2[1,4′]bipiperidinyl-1′-yl-1-(6methoxy-pyridin-3-ylmethyl)-2-oxo-et-hyl]-amide;
(±)-4-(2-Oxo-1,4-dihydro-2H-quinazolin-3-yl)-piperidine-1-carboxylic acid [2-[1,4′]bipiperidinyl-1′-yl-1-(2-methoxy-pyrimidin-5-ylmethyl)-2-ox-o-ethyl]-amide;
(±)-4-(2-Oxo-1,4-dihydro-2H-quinazolin-3-yl)-piperidine-1-carboxylic acid [(1-(6-benzyloxy-pyridin-3-ylmethyl)-2-[1,4′bipiperidinyl-1′-yl-2-ox-o-ethyl]-amide;
(±)-4-(2-Oxo-1,4-dihydro-2H-quinazolin-3-yl)-piperidine-1-carboxylic acid [2-[1,4′]bipiperidinyl-1′-yl-2-oxo-1-(6-oxo-1,6-dihydro-pyridin-3-yl-methyl)-ethyl]-amide;
(±)-2-(7-Methyl-1H-indazol-5-ylmethyl)-4-oxo-4-[4-(2-oxo-1,4-dihydro-2-H-quinazolin-3-yl)-piperidin-1-yl]-butyric acid methyl ester;
(±)-1-[1,4′]Bipiperidinyl-1′-yl-2-(7-methyl-1H-indazol-5-ylmethyl)-4-[-4-(2-oxo-1,4-dihydro-2H-quinazolin-3-yl)-piperidin-1-yl]butane-1,4-dione;
(±)-1-(1,4-Dioxa-8-aza-spiro[4.5]dec-8-yl)-2-(7-methyl-1H-indazol-5-yl-methyl)-4-[4-(2-oxo-1,4-dihydro-2H-quinazolin-3-yl)-piperidin-1-yl]-butane-1,4-dione;
(±)—N,N-Dimethyl-2-(7-methyl-1H-indazol-5-ylmethyl)-4-oxo-4-[4-(2-oxo-1,4-dihydro-2H-quinazolin-3-yl)-piperidin-1-yl]-butyramide;
(±)-1-(2,6-Dimethyl-morpholin-4-yl)-2-(7-methyl-1H-indazol-5-ylmethyl)-4-[4-(2-oxo-1,4-dihydro-2H-quinazolin-3-yl)-piperidin-1-yl]-butane-1,4-dione;
(±)-2-(7-Methyl-1H-indazol-5-ylmethyl)-1-(4-methyl-piperidin-1-yl)-4-[-4-(2-oxo-1,4-dihydro-2H-quinazolin-3-yl)-piperidin-1-yl]-butane-1,4-dione;
(±)-2-(7-Methyl 1H-indazol-5-ylmethyl)-1-morpholin-4-yl-4-[4-(2-oxo-1,4-dihydro-2H-quinazolin-3-yl)-piperidin-1-yl]-butane-1,4-dione;
(±)-2-(7-Methyl-1H-indazol-5-ylmethyl)-4-[4-(2-oxo-1,4-dihydro-2H-quinazolin-3-yl)-piperidin-1-yl]-1-piperidin-1-yl-butane-1,4-dione;
(±)-1-(1,4-Dioxa-8-aza-spiro[4.5]dec-8-yl)-2-(1H-indazol-5-ylmethyl)-4-[4-(2-oxo-1,4-dihydro-2H-quinazolin-3-yl)-piperidin-1-yl]-butane-1,4-dione;
(±)-2-(1H-Indazol-5-ylmethyl)-N,N-dimethyl-4-oxo-4-[4-(2-oxo-1,4-dihydro-2H-quinazolin-3-yl)-piperidine-1-yl]-butyramide;
(±)-5-{2-([1,4′]Bipiperidinyl-1′-carbonyl)-4-oxo-4-[4-(2-oxo-1,4-dihydro-2H-quinazolin-3-yl)-piperidin-1-yl]-butyl}-indazole-1-carboxylic acid tert-butyl ester; (±)-2-(7-Methyl-1H-indazol-5-ylmethyl)-4-oxo-4-[4-(2-oxo-1,4-dihydro-2-H-quinazolin-3-yl)-piperidin-1yl]-N-prop-2-ynyl-butyramide;
(L)-{1-([1,4′]Bipiperidinyl-1′-carbonyl)-3-oxo-3-[4-(2-oxo-1,4-dihydro-2H-quinazolin-3-yl)-piperidin-1-yl]-propyl}-carbamic acid tert-butyl ester;
(L)-1-[1,4′]Bipiperidinyl-1′-yl-2-(1H-indol-5-ylamino)-4-[4-(2-oxo-1,4-di-hydro-2H-quinazolin-3-yl)-piperidin-1-yl]-butane-1,4-dione;
(L)-1-[1,4′]Bipiperidinyl-1′-yl-2-(5-chloro-2-nitro-phenylamino)-4-[4-(2-oxo-1,4-dihydro-2H-quinazolin-3-yl)-piperidin-1-yl]-butane-1,4-dione;
(L)-1-[1,4′Bipiperidinyl-1′-yl-2-(6-chloro-pyrimidin-4-ylamino)-4-[4-(2-o-xo-1,4-dihydro-2H-quinazolin-3-yl)-piperidin-1-yl]-butane-1,4-dione;
(L)-1-[1,4′]Bipiperidinyl-1′-yl-2-(2-chloro-9H-purin-6-ylamino)-4-[4-(2-o-xo-1,4-dihydro-2H-quinazolin-3-yl)-piperidin-1-yl]-butane-1,4-dione;
(L)-2-(4-Amino-6-methyl-5-nitro-pyrimidin-2-ylamino)-1-[1,4′]bipiperidiny-I-1′-yl-4-[4-(2-oxo-1,4-dihydro-2H-quinazolin-3-yl)-piperidin-1-yl]-butane-1,4-dione;
(L)-1-[1,4′]Bipiperidinyl-1′-yl-2-(4,5-diamino-6-methyl-pyrimidin-2-ylamino)-4-[4-(2-oxo-1,4-dihydro-2H-quinazolin-3-yl)-piperidin-1-yl]-butane-1,4-dione;
(L)-1-[1,4′]Bipiperidinyl-1′-yl-2-(7-methyl-1H-[1,2,3]triazo[4,5-d]pyrimidin-5-ylamino)-4-[4-(2-oxo-1,4-dihydro-2H-quinazolin-3-yl)-piperidin-1-yl]-butane-1,4-dione;
(L)-1-[1,4′]Bipiperidinyl-1′-yl-2-((2′-pyridyl)-methyl-amino)-4-]4-(2-oxo-1,4-dihydro-2H-quinazolin-3-yl)-piperidin-1-yl]-butane-1,4-dione;
(L)-1-[1,4′]Bipiperidinyl-1′-yl-2-((5′-indazolyl)-methyl-amino)-4-[4-(2-o-xo-1,4-dihydro-2H-quinazolin-3-yl)-piperidin-1-yl]-butane-1,4-dione;
(L)-1-[1,4′]Bipiperidinyl-1′-yl-2-((3′-methyl-phenyll)-methyl-amino)-4-[4-(2-oxo-1,4-dihydro-2H-quinazolin-3-yl)-piperidin-1-yl]-butane-1,4-dione;
(L)-1-[1,4′]Bipiperidinyl-1′-yl-4-[4-(2-oxo-1,4-dihydro-2H-quinazolin-3-y-I)-piperidin-1-yl]-2-(pyrimidin-4-ylamino)-butane-1,4-dione;
(L)-1-[1,4′]Bipiperidinyl-1′-yl-2-(4-hydroxy-cyclohexylamino-4-[4-(2-oxo-1,4-dihydro-2H-quinazolin-3-yl)-piperidin-1-yl]-butane-1,4-dione;
(L)-1-[1,4′]Bipiperidinyl-1′-yl-2-[(1H-imidazol-4-ylmethyl)-amino]-4-[4-(-2-oxo-1,4-dihydro-2H-quinazolin-3-yl)piperidin-1-yl]-butane-1,4-dione;
(L)-N-{1-([1,4′]Bipiperidinyl-1′-carbonyl)-3-oxo-3-[4-(2-oxo-1,4-dihydro-2H-quinazolin-3-yl)-piperidin-1-yl]-propyl}-4-hydroxy-benzamide;
(L)-N-{1-([1,4′]Bipiperidinyl-1′-carbonyl)-3-oxo-3-[4-(2-oxo-1,4-dihydro-2H-quinazolin-3-yl)-piperidin-1-yl]-propyl}-4-hydroxy-benzamide;
(L)-1H-Pyrazole-3-carboxylic acid{1-([1,4′]bipiperidinyl-1′-carbonyl)-3-oxo-3-[4-(2-oxo-1,4-dihydro-2H-quinazolin-3-yl)-piperidin-1-yl]-propyl}-amide;

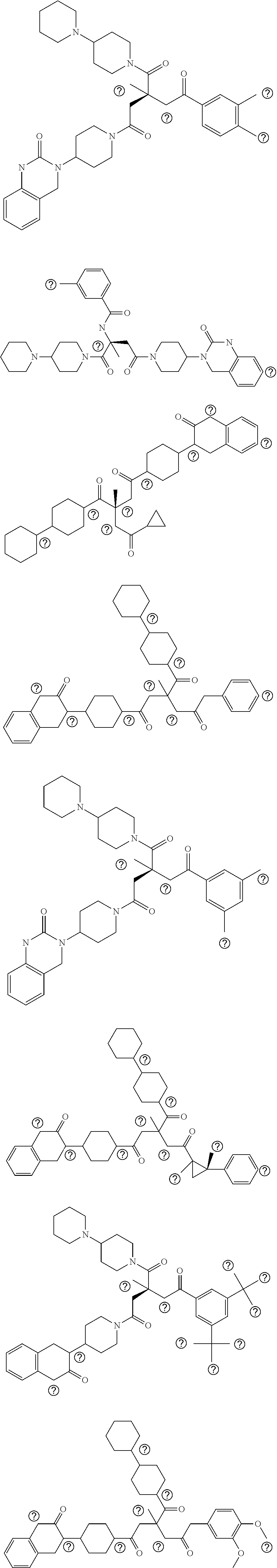

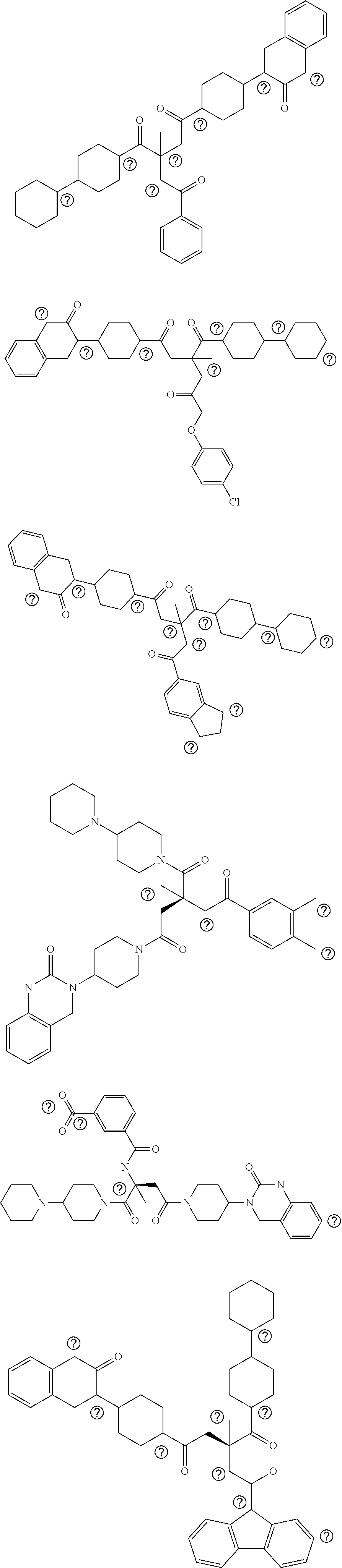

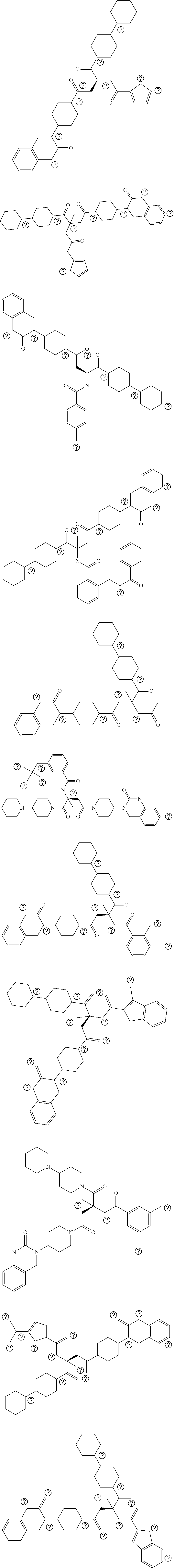

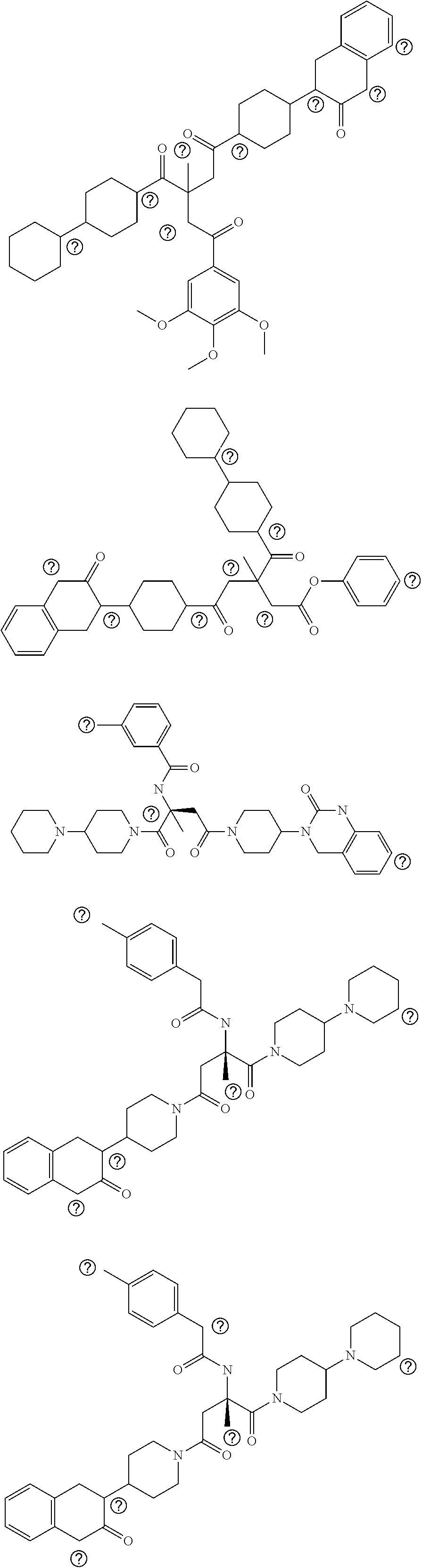

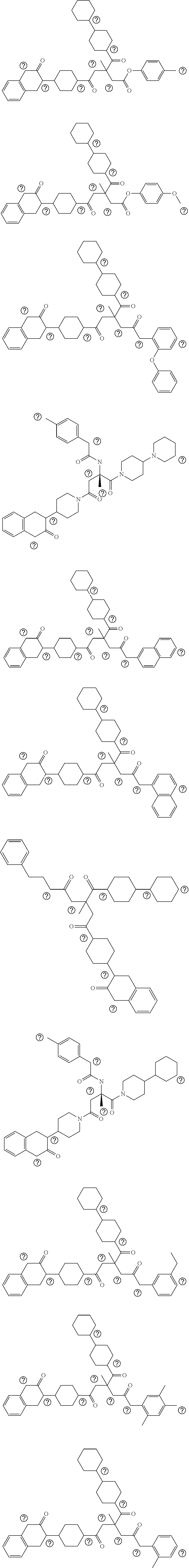

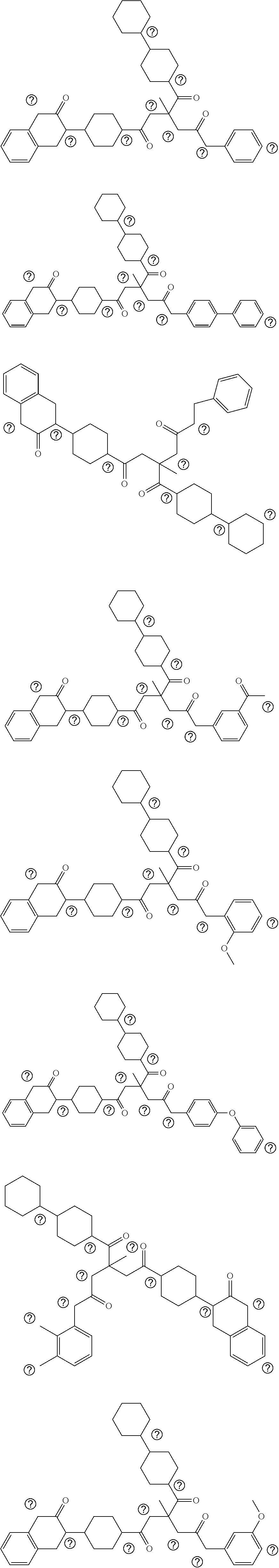

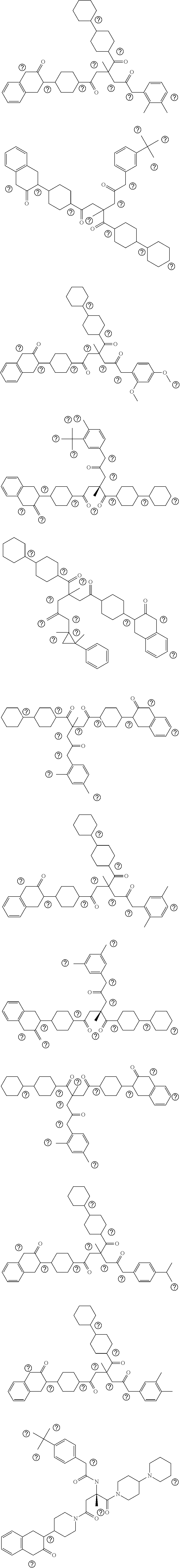

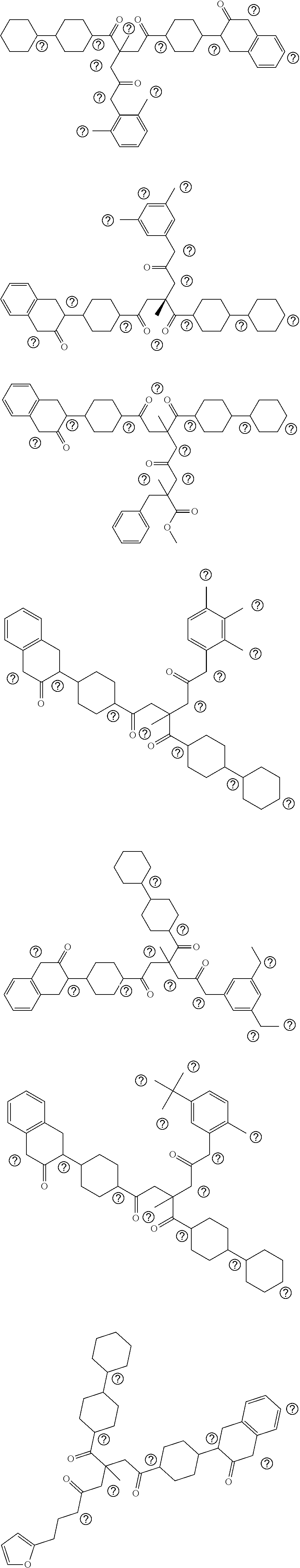

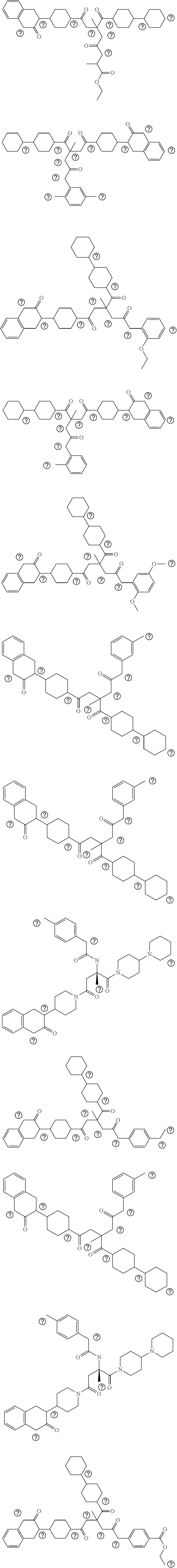

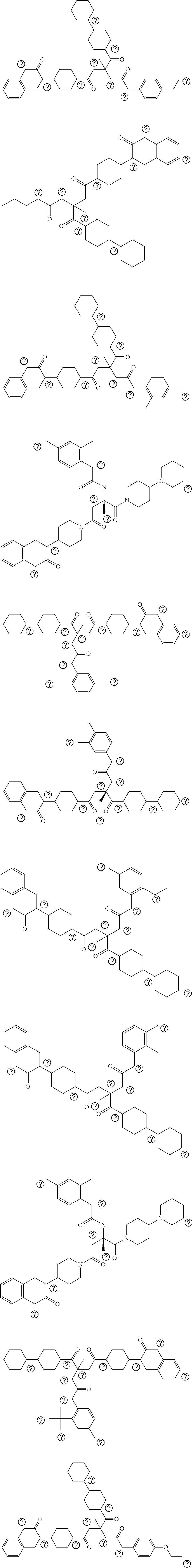

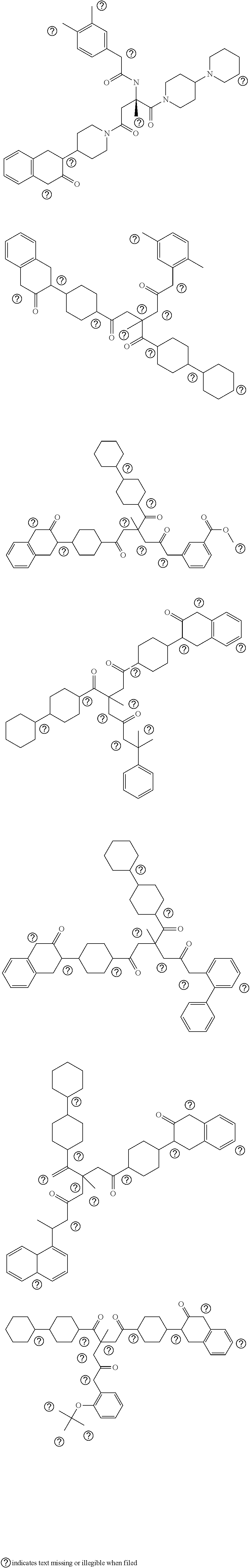

(±)-1-[1,4′]Bipiperidinyl-1′-yl-2-(1H-indazol-5-ylamino)-4-[4-(2-oxo-1-,4-dihydro-2H-quinazolin-3-yl)-piperidin-1-yl]-butane-1,4-dione;
(±)-3-(3-Cyano-1H-indol-5-yl)-2-{[4-(2-oxo-1,4-dihydro-2H-quinazolin-3-yl)-piperidin-1-carbonyl]-amino}-propionic acid methyl ester;
(±)-4-(2-Oxo-1,4-dihydro-2H-quinazolin-3-yl)-piperidine-1-carboxylic acid [2-[1,4′]bipiperidinyl-1′-yl-1-(3-cyano-1H-indol-5-yl-methyl)-2-oxo-ethyl]-amide;
(±)-4-(2-Oxo-1,4-dihydro-2H-quinazolin-3-yl)-piperidine-1-carboxylic acid [2-[1,4′]bipiperidinyl-1′-yl-1 (3-cyano-7-methyl-1H-indol-5-yl-methyl-)-2-oxo-ethyl]-amide;
(±)-3-(7-Isopropyl-1H-indazol-5-yl)-2-{[4-(2-oxo-1,4-dibydro-2H-quinazolin-3-yl)-piperidin-1-carbonyl]-amino}-propionic acid methyl ester;
(±)-4-(2-Oxo-1,4-dihydro-2H-quinazolin-3-yl)-piperidine-1-carboxylic acid [2-[1,4′]bipiperidinyl-1′-yl-1 (7-isopropyl-1H-indazol-5-yl-methyl)-2-oxo-ethyl]-amide;
(±)-4-(2-Oxo-1,4-dihydro-2H-quinazolin-3-yl)-piperidine-1-carboxylic acid [2-[1,4′]bipiperidinyl-1′-yl-1(7-ethyl-1H-indazol-5-yl-methyl)-2-oxo-1-ethyl]-amide; (±)-4-(2,2-Dioxo-1,4-dihydro-2H-2.lamda..sup.6-benzo[1,2,6]thiadiazin-3-yl)-piperidine-1-carboxylic acid [2-[1,4′]bipiperidinyl-1′-yl-1(7-methyl-1H-indazol-5-yl-methyl)-2-oxo-ethyl]-amide;
(±)-4-(2,2-Dioxo-1,4-dihydro-2H-2.lamda..sup.6-benzo[1,2,6]t-hiadiazin-3-yl)-piperidin-1-carboxylic acid [2-[1,4′bipiperidinyl-1′-yl-1(7-ethyl-3-methyl-1H-indazol-5-yl-methyl)-2-oxo-ethyl]-amide;
(±)-2-[4-(6-Cyano-2-oxo-1,4-dihydro-2H-qinazolin-3-yl)piperidine-1-carbonyl]-amino]-3-(7-methyl-1H-indazol-5-yl)-propionic acid methyl ester;
(±)-4-(6-Cyano-2-oxo-1,4-dihydro-2H-qinazolin-3-yl)-piperidine-1-carboxylic acid{2-[1,4′bipiperidinyl-1′-yl-1-(7-methyl-1H-indazol-5-yl methyl)-2-oxo-ethyl}-amide;
(±)-4-(2-Oxo-1,2,4,5-tetrahydro-benzo[d][1,3]diazepin-3-yl-1-carboxylic acid{2-[1,4′]bipiperidinyl-1′-yl-1-(7-methyl-1H-indazol-5-yl methyl)-2-oxo-ethyl}-amide;
(±)-4-(6-Hydroxy-2-oxo-1,4-dihydro-2H-qinazolin-3-yl)-piperidine-1-carboxylic acid{2-[1,4′]bipiperidinyl-1′-yl-1-(7-methyl-1H-indazol-5-yl methyl)-2-oxo-ethyl}-amide;
(±)-4-(8-Methoxy-2-oxo-1,4-dihydro-2H-qinazolin-3-yl)-piperidine-1-carboxylic acid{2-[1,4′]bipiperidinyl-1′-yl-1-(7-methyl-1H-indazol-5-yl methyl)-2-oxo-ethyl}-amide;
(±)-4(8-Chloro-2-oxo-1,4-dihydro-2H-qinazolin-3-yl)-piperidine-1-carboxylic acid{2-[1,4′]bipiperidinyl-1′-yl-1-(7-methyl-1H-indazol-5-yl methyl)-2-oxo-ethyl}-amide;
(±)—N-(3-(7-Ethyl-1H-indazol-5-yl)-1-(6,7-dihydro-1H-pyrazolo[4,3-c]py-ridin-5(4H)-yl)-1-oxopropan-2-yl)-4-(1,2-dihydro-2-oxoquinazolin-3(4H)-yl)-piperidine-1-carboxamide;
(±)-Methyl 2-(4-(8-fluoro-1,2-dihydro-2-oxoquinazolin-3(4H)-yl)piperidine-1-carboxamide)-3-(7-methyl-1H-indazol-5-yl)propanoate;
(±)-4-(8-Fluoro-1,2-dihydro-2-oxoquinazolin-3(4H)-yl)-N-(3-(7-methyl-1-H-indazol-5-yl)-1-oxo-1-(4-(piperidin-1-yl)piperidin-1-yl)propan-2-yl)piperidine-1-carboxamide;
(±)4-(8-Fluoro-1,2-dihydro-2-oxoquinazolin-3(4H)-yl)-N-(3-(7-methyl-1H-indazol-5-yl)-1-oxo-1-(4-phenylpiperazin-1-yl)propan-2-yl)piperidine-1-carboxamide;
(±)-4-(8-Fluoro-1,2-dihydro-2-oxoquinazolin-3(4H)-yl)-N-(1-(-4-(4-fluorophenyl)piperazin-1-yl)-3-(7-methy-1H-indazol-5-yl)-1-oxopropan-2-yl)piperidine-1-carboxamide;
(±)-4-(8-Fluoro-1,2-dihydro-2-oxoquinazolin-3(4H)-yl)-N-(1-(4-(2-fluor-ophenyl)piperazin-1-yl)-3-(7-methyl-1H-indazol-5-yl)-1-oxopropan-2-yl)piperidine-1-carboxamide;
(±)-4-(8-Fluoro-1,2-dihydro-2-oxoquinazolin-3(4H)-yl)-N-(3-(7-methyl-1-H-indazol-5-yl)-1-oxo-1-(4-o-tolylpiperazin-1-yl)propan-2-yl)piperidine-1-carboxamide;
(±)-Methyl 2-(4-(8-fluoro-1,2-dihydro-2-oxoquinazolin-3(4H)-yl)piperidine-1-carboxamide)-3-(7-ethyl-3-methyl-1H-indazol-5-yl)propanoate;
(±)—N-(3-(7-Ethyl-3-methyl-1H-indazol-5-yl)-1-oxo-1-(4-(piperidin-1-yl)piperidin-1-yl)propan-2-yl)-4-(8-fluoro-1,2-dihydro-2-oxoquinazolin-3(4H)-yl)piperidine-1-carboxamide;
(R)-N—((R)-3-(2-Oxo-2,3-dihydro-benzooxazol-6-yl)-1-oxo-1-(4-(piperidin-1-yl)propan-2-yl)-4-(8-fluoro-1,2-dihydro-2-oxoquinazolin-3(4H)-yl)piperidine-1-carboxamide;
(±)-4-(8-Fluoro-1,2-dihydro-2,4-dioxoquinazolin-3(4H)-yl)-N-(3-(7-methyl-1H-indazol-5-yl)-1-oxo-1-(4-(piperidin-1-yl)piperidin-1-yl)propan-2-yl)-piperidine-1-carboxamide;
N—((R)-3-(2-(trifluoromethyl)-1H-benzo[d]imidazol-5-yl)-1-oxo-1-(4-(piperidin-1-yl)piperidin-1-yl)propan-2-yl)-4-(1,2-dihydro-2-oxoquinazolin-3(4H)-yl)piperidine-1-carboxamide;
N—((R)-1-(dimethylcarbamoyl)-2-(2-trifluoromethyl)-1H-benzo[d]imidazol-5-yl)ethyl)-4-(1,2-dihydro-2-oxoquinazolin-3(4H)-yl)piperidine-1-carboxamide-;
(R)-methyl 2-(4-(1,2-dihydro-2-oxoquinazolin-3(4H)-yl)piperidine-1-carboxamide)-3-(2-,3-dihydro-2-oxo-1H-benzo[d]imidazol-6-yl)propanoate;
N—((R)-3-(2,3-dihydro-2-oxo-1H-benzo[d]imidazol-6-yl)-1-oxo-1(4-piperidine-1-yl)propan-2-yl)-4-(1,2-dihydro-2-oxoquinazolin-3(4H)-yl)piperidine-1-carboxamide;
N—((R)-1-(dimnethylcarbamoyl)-2-(2,3-dihydro-2-oxo-1H-benzo[d]imidazol-6-yl)ethyl)-4-(1,2-dihydro-2-oxoquinazolin-3(4H))-yl)piperidine-1-carboxamide;
4-(1,2-dihydro-2,4-dioxoquinazolin-3-(4H)-yl)-N—((R)-3-(7-methyl-1H-benzo[d][1,2,3]triazol-5-yl-1-oxo-1-(4-(piperidin-1-yl)piperidin-1-yl)propan-2-yl)piperidine-1-carboxamide;
(R)-4-(2-Oxo-1,4-dihydro-2H-quinazolin-3-yl)-piperidine-1-carboxylic acid[2-(4-cyclohexyl-piperazin-1-yl)-2-oxo-1-(2-oxo-2,3-dihydro-benzooxaz-ol-6-ylmethyl)-ethyl]-amide;
(R)-4-(2-Oxo-1,4-dihydro-2H-quinazolin-3-yl)-piperidine-1-carboxylic acid[2-(4-isopropyl-piperazin-1-yl)-2-oxo-1-(2-oxo-2,3-dihydro-benzooxazol-6-ylmethyl)-ethyl]-amide;
(R)-4-(2-Oxo-1,4-dihydro-2H-quinazolin-3-yl)-piperidine-1-carboxylic acid[2-[1,4′]bipiperidinyl-1′-yl-1-(4-chloro-2-oxo-2,3-dihydro-benzooxazol-6-ylmethyl)-2-oxo-ethyl]-amide;
(R)-4-(2-Oxo-1,4-dihydro-2H-quinazolin-3-yl)-piperidine-1-carboxylic acid[2-[1,4′]bipiperidinyl-1′-yl-1-(5-chloro-2-oxo-2,3-dihydro-benzooxazol-6-ylmethyl)-2-oxo-ethyl]-amide;
(R)-4-(2-Oxo-1,4-dihydro-2H-quinazolin-3-yl)-piperidine-1-carboxylic acid[2-[1,4′]bipiperidinyl-1′-yl-1-(4-bromo-2-oxo-2,3-dihydro-benzooxazol-6-ylmethyl)-2-oxo-ethyl]-amide;
(R)-4-(2-Oxo-1,4-dihydro-2H-quinazolin-3-yl)-piperidine-1-carboxylic acid[2-[1,4′]bipiperidinyl-1′-yl-1-(5-bromo-2-oxo-2,3-dihydro-benzooxazol-6-ylmethyl)-2-oxo-ethyl]-amide;
(R)-4-(2-Oxo-1,4-dihydro-2H-quinazolin-3-yl)-piperidine-1-carboxylic acid[2-[1,4′]bipiperidinyl-1′-yl-1-(4-iodo-2-oxo-2,3-dihydro-benzooxazol-6-ylmethyl)-2-oxo-ethyl]-amide;
(±)—N-(1-Benzyl-2-hydroxy-ethyl)-2-(7-methyl-1H-indazol-5-ylmethyl)-4-oxo-4-[4-(2-oxo-1,4-dihydro-2H-quinazolin-3-yl)-piperidin-1-yl]-butyramide-;
(4Phenyl-acetic acid N′-{2-(7-methyl-1H-indazol-5-ylmethyl)-4-oxo-4-[4-(2-oxo-1,4-dihydro 2H-quinazolin-3-yl)-piperidin-1-yl)-butyryl}-hydrazide;
(±)-1-[1,4′]Bipiperidinyl-1′-yl-4-[4-(8-fluoro-2-oxo-1,4-dihydro-2H-quinazolin-3-yl)-piperidin-1-yl]-2-(7-methyl-1H-indazol-5-ylmethyl)-butane-1-,4-dione;
(±)1-(4-Cyclohexyl-piperazin-1-yl)-2-(2-oxo-2,3-dihydro-benzo-oxazol-6-ylmethyl)-4-[4-(2-oxo-1,4-dihydro-2H-quinazolin-3-yl)-piperidin-1-yl]-butane-1,4-dione;
(±)-4-(2-Oxo-1,4-dihydro-2H-quinazolin-3-yl)piperidine-1-carboxylic acid[2-(4-cyclohexyl-piperazin-1-yl)-1-(7-methyl-1H-indazol-5-ylmethyl)-2-oxo-ethyl]-amide;
(±)-4-(2-Oxo-1,4-dihydro-2H-quinazolin-3-yl)-piperidine-1-carboxylic acid[2-[4-(4-fluoro-phenyl)-piperazin-1-yl]-1-(7-methyl-1H-indazol-5-ylmethyl)-2-oxo-ethyl]-amide;
(±)-3-(7-Methyl-1H-indazol-5-yl)-2-{[4-(2-oxo-1,4-dihydro-2H-quinazolin-3-yl)-piperidine-1-carbonyl]-amino)}-propionic acid tert-butyl ester;
(±)-3-(7-Methyl-1H-indazol-5-yl)-2-}[4-(2-oxo-1,4-dihydro-2H-quinazolin-3-yl)-piperidine-1-carbonyl]-amino}-propionic acid 1-methyl cyclohexyl ester;
(±)-3-(7-Methyl-1H-indazol-5-yl)-2-{[4-(2-oxo-1,4-dihydro-2H-quinazolin-3-yl)-piperidine-1-carbonyl}-amino}-propionic acid 1-aza-bicyclo[2.2.2]oct-3-yl ester;
(±)-3-(7-Methyl-1H-indazol-5-yl)-2-{[4-(2-oxo-1,4-dihydro-2H-quinazolin-3-yl)-piperidine-1-carbonyl]-amino}-propionic acid piperidin-4-yl ester;
(±)-4-(3-(7-Methyl-1H-indazol-5-yl)-2-{[4-(2-oxo-1,4-dihydro-2H-quinazolin-3-yl)-piperidine-1-carbonyl[-amino}-propionyloxy)-piperidine-1-carboxylic acid tert-butyl ester,
(±)-3-(7-Methyl-1H-indazol-5-yl)-2-{[4-(2-oxo-1,4-dihydro-2H-quinazolin-3-yl)-piperidine-1-carbonyl]-amino}-propionic acid 3,4,5,6-tetrahydro-2H-[1,4′]bipyridinyl-4-yl ester,
(±)-3-(7-Methyl-1H-indazol-5-yl)-2-{[4-(2-oxo-1,4-dihydro-2H-quinazolin-3-yl)-piperidine-1-carbonyl]-amino)-propionic acid 1-diethylamino-1-methyl-ethyl ester;
(±)-3-(7-Methyl-1H-indazol-5-yl)-2-{[4-(2-oxo-1,4-dihydro-2H-quinazolin-3-yl)-piperidine-1-carbonyl]-amino}-propionic acid 1,1-dimethyl-2-phenyl-ethyl ester;
(±)-3-(7-Methyl-1H-indazol-5-yl)-2-{[4-(2-oxo-1,4-dihydro-2H-quinazolin-3-yl)-piperidine-1-carbonyl]-amino)-propionic acid 1,1-dimethyl-3-phenyl-propyl ester;
(±)-3-(7-Methyl-1H-indazol-5-yl)-2-{[4-(2-oxo-1,4-dihydro-2H-quinazolin-3-yl)-piperidine-1-carbonyl]-amino}-propionic acid ethyl ester; and
(R)-4-(2-Oxo-1,4-dihydro-2H-quinazolin-3-yl)-piperidine-1d-carboxylic acid[2-[1,4′]bipiperidinyl-1′-yl-1-(7-methyl-1H-indazol-5-ylmethyl)-2-oxo-1-ethyl]amide.

In some embodiments, the CGRP receptor antagonist may be Zavegepant, which has the formula (R)-N-(3-(7-methyl-1H-indazol-5-yl)-1-(4-(1-methylpiperidin-4-yl)piperazin-1-yl)-1-oxopropan-2-yl)-4-(2-oxo-1,2-dihydroquinolin-3-yl)piperidine-1-carboxamide, and which has the following structure

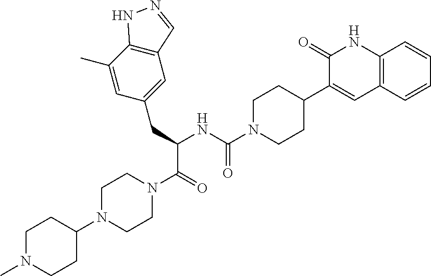 [SMILES: Cc1cc(C[C@@H](NC(=O)N2CCC(c3cc4ccccc4[nH]c3=O)CC2)C(=O)N2CCN(C3CCN(C)CC3)CC2)cc2cn[nH]c12]

or a pharmaceutically acceptable salt thereof.

### Formula III

In some embodiments, a method of treating, ameliorating, alleviating, providing for prophylaxis or prevention of, halting the progression of, and/or reducing the risk of trigeminal neuralgia, may comprise administering to a subject in need thereof a CGRP receptor antagonist according to Formula (III):

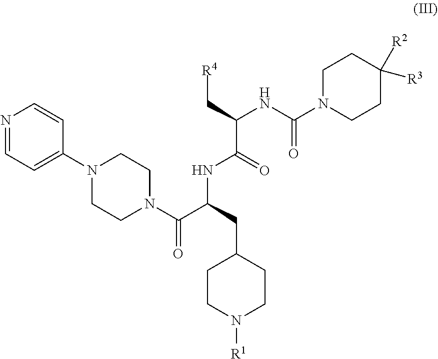

or a salt thereof or an optical isomer thereof, wherein
R¹ is H or Q-(C₁-C₆)alkyl; where Q is a bond, C(O) or C(O)O and where the (C₁-C₆)alkyl can be optionally substituted by N(C₁-C₃alkyl)₂ or CO₂H;
R² is H or forms a spirocyclic heterocyclic ring with R³;
R³ forms a spirocyclic heterocyclic ring with R² or is a heterocyclic ring if R² is H; and
R⁴ is an optionally substituted aryl group which may be monocyclic or fused to a further ring.

In some embodiments, the CGRP receptor antagonists described by general Formula III may be selected from any of the following specific formulas:
N-[(2R)-3-(7-methyl-1H-indazol-5-yl)-1-oxo-1-({(2S)-1-oxo-3-(piperidin-4-yl)-1-[4-(pyridin-4-yl)piperazin-1-yl]propan-2-yl}amino)propan-2-yl]-4-(2-oxo-2,3-dihydro-1H-imidazo[4,5-b]pyridin-1-yl)piperidine-1-carboxamide;
tert-butyl 4-{(2S)-2-{[(2R)-3-(7-methyl-1H-indazol-5-yl)-2-({[[4-(2-oxo-1,2-dihydroquinolin-3-yl)piperidin-1-yl]carbonyl}amino)propanoyl]amino}-3-oxo-3-[4-(py-ridin-4-yl)piperazin-1-yl]propyl}piperidine-1-carboxylate;
N-[(2R)-3-(7-methyl-1H-indazol-5-yl)-1-oxo-1-({(2S)-1-oxo-3-(piperidin-4-yl)-1-[4-(pyridin-4-yl)piperazin-1-yl]propan-2-yl}amino)propan-2-yl]-4-(2-oxo-1,2-dihydroquinolin-3-yl)piperidine-1-carboxamide;
N-[(2R)-3-(7-methyl-1H-indazol-5-yl)-1-oxo-1-({(2S)-1-oxo-3-(1-propylpiperidin-4-yl)-1-[4-(pyridin-4-yl)piperazin-1-yl]propan-2-yl}amino)propan-2-yl]-4-(2-oxo-1,2-dihydroquinolin-3-yl)piperidine-1-carboxamide;
3,5-dibromo-Na—{[4-(2-oxo-1,2-dihydroquinolin-3-yl)piperidin-1-yl]carbonyl}-N-{(2S)-1-oxo-3-(piperidin-4-yl)-1-[4-(pyridin-4-yl)piperazin-1-yl]propan-2-yl}-D-tyrosinamide;
N-[(2R)-3-(7-methyl-1H-indazol-5-yl)-1-oxo-1-({(2S)-1-oxo-3-(1-pentanoylpiperidin-4-yl)-1-[4-(pyridin-4-yl)piperazin-1-yl]propan-2-yl}amino)propan-2-yl]-4-(2-oxo-1,2-dihydroquinolin-3-yl)piperidine-1-carboxamide;
N-[(2R)-1-({(2S)-3-(1-ethylpiperidin-4-yl)-1-oxo-1-[4-(pyridin-4-yl)piperazin-1-yl]propan-2-yl}amino)-3-(7-methyl-1H-indazol-5-yl)-1-oxopropan-2-yl-]-4-(2-oxo-1,2-dihydroquinolin-3-yl)piperidine-1-carboxamide;
3,5-dibromo-Na—{[4-(2-oxo-2,3-dihydro-1H-imidazo[4,5-b]pyridin-1-yl)piperidin-1-yl]carbonyl}-N-{(2S)-1-oxo-3-(piperidin-4-yl)-1-[4-(pyridin-4-yl)piperazin-1-yl]propan-2-yl}-D-tyrosinamide;
3,5-dibromo-Na—{[4-(2-oxo-1,4-dihydroquinazolin-3(2H)-yl)piperidin-1-yl]carbonyl}-N-{(2S)-1-oxo-3-(piperidin-4-yl)-1-[4-(pyridin-4-yl)piperazin-1-yl]propan-2-yl}-D-tyrosinamide;
N-[(2R)-3-(7-methyl-1H-indazol-5-yl)-1-oxo-1-({(2S)-1-oxo-3-(piperidin-4-yl)-1-[4-(pyridin-4-yl)piperazin-1-yl]propan-2-yl}amino)propan-2-yl]-4-(2-oxo-1,4-dihydroquinazolin-3(2H)-yl)piperidine-1-carboxamide;
N-[(2R)-3-(7-methyl-1H-indazol-5-yl)-1-oxo-1-({(2S)-1-oxo-3-(piperidin-4-yl)-1-[4-(pyridin-4-yl)piperazin-1-yl]propan-2-yl}amino)propan-2-yl]-2′-oxo-1′,2′-dihydro-1H-spiro[piperidine-4,4′-pyrido[2,3-d][1,3]oxazine]-1-carboxamide;
N-[(2R)-1-({(2S)-3-{1-[2-(dimethylamino)ethyl]piperidin-4-yl}-1-oxo-1-[4-(pyridin-4-yl)piperazin-1-yl]propan-2-yl}amino)-3-(7-methyl-1H-ind-azol-5-yl)-1-oxopropan-2-yl]-4-(2-oxo-2,3-dihydro-1H-imidazo[4,5-b]pyridin-1-yl)piperidine-1-carboxamide;
3-(4-{(2S)-2-{[(2R)-3-(7-methyl-1H-indazol-5-yl)-2-({[4-(2-oxo-2,3-dihydro-1H-imidazo[4,5-b]pyridin-1-yl)piperidin-1-yl]carbonyl}amino)propanoyl]am-ino}-3-oxo-3-[4-(pyridin-4-yl)piperazin-1-yl]propyl}piperidin-1-yl)-3-oxop-ropanoic acid, ammonium salt; and
3, 5-dibromo-Nα-[(2′-oxo-1′,2′-dihydro-1H-spiro[piperidine-4,4′-pyrido-[2,3-d][1,3]oxazin]-1-yl)carbonyl]-N-{(2S)-1-oxo-3-(piperidin-4-yl)-1-[4-(-pyridin-4-yl)piperazin-1-yl]propan-2-yl}-D-tyrosinamide;
   or a salt thereof or an optical isomer thereof.

In some embodiments, the CGRP receptor antagonist may be BHV-3100, which has the formula N-[(2R)-3-(7-methyl-1H-indazol-5-yl)-1-oxo-1-({(2S)-1-oxo-3-(piperidin-4-yl)-1-[4-(pyridin-4-yl)piperazin-1-yl]propan-2-yl}amino)propan-2-yl]-2′-oxo-1′,2′-dihydro-1H-spiro[piperidine-4,4′-pyrido[2,3-d][1,3]oxazine]-1-carboxamide, and which has the following structure:

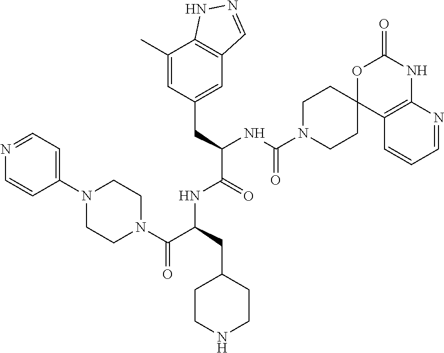 [SMILES: Cc1cc(C[C@@H](NC(=O)N2CCC3(CC2)OC(=O)Nc2ncccc23)C(=O)N[C@@H](CC2CCNCC2)C(=O)N2CCN(c3ccncc3)CC2)cc2cn[nH]c12]

or a pharmaceutically acceptable salt thereof.

### Pharmaceutical Compositions and Methods of Treatment

The compounds of Formula I, Formula II, and/or Formula III inhibit the CGRP receptor. As such, they are useful for treating conditions or disorders associated with aberrant CGRP levels or where modulating CGRP levels may have therapeutic benefit.

Accordingly, another aspect of the disclosure is a pharmaceutical composition comprising a compound of Formula I with a pharmaceutically acceptable adjuvant, carrier, or diluent.

Accordingly, another aspect of the disclosure is a pharmaceutical composition comprising a compound of Formula II with a pharmaceutically acceptable adjuvant, carrier, or diluent.

Accordingly, another aspect of the disclosure is a pharmaceutical composition comprising a compound of Formula III with a pharmaceutically acceptable adjuvant, carrier, or diluent.

Compounds are generally given as pharmaceutical compositions comprised of a therapeutically effective amount of one or more of a compound of Formula I, Formula II, and/or Formula III, or pharmaceutically acceptable salt(s) thereof, and a pharmaceutically acceptable carrier, further optionally containing conventional excipients. A therapeutically effective amount is the amount needed to provide a meaningful patient benefit as determined by practitioners in that art. Pharmaceutically acceptable carriers are those conventionally known carriers having acceptable safety profiles. Compositions encompass all common solid and liquid forms including capsules, tablets, losenges, and powders as well as liquid suspensions, syrups, elixers, and solutions. Solid compositions may by formed in timed or sustained released formulations. Compositions are made using common formulation techniques and conventional excipients (such as binding and wetting agents) and vehicles (such as water and alcohols).

The disclosure and methods encompass all conventional modes of administration including oral, parenteral, intranasal, sublingual, topical, and transdermal methods. Typically, the daily dose may be 0.01-100 mg/kg body weight daily. Generally, more compound is required orally and less parenterally. The specific dosing regime, however, should be determined by a physician using sound medical judgement.

Methods of treatment as disclosed herein are broadly directed to treatment of pain, such as neuralgia, neuropathy, and neuropathic pain, in a patient in need thereof with one or more CGRP receptor antagonists according to Formulas (I), (II), or (III). A patient may have one or more types of pain, at least one of which may benefit from administration of a GRP receptor antagonist. Some methods of treatment specifically recite neuralgia, such as trigeminal neuralgia, and are broadly applicable to any pain which may benefit from administration of a CGRP receptor antagonist.

Among other routes of administration, the standard routes of administration described by the FDA are contemplated herein as shown in Table 1 below (FDA Routes of Administration; retrieved from www.fda.gov; content current as of Nov. 14, 2017).

TABLE 1
FDA Routes of Administration

| | | | | NCI |
|---|---|---|---|---|
| | | SHORT | FDA | CONCEPT |
| NAME | DEFINITION | NAME | CODE | ID |
| AURICULAR (OTIC) | Administration to or by way of the | OTIC | 013 | C38192 |
| | ear. | | | |
| BUCCAL | Administration directed toward the | BUCCAL | 030 | C38193 |
| | cheek, generally from within the | | | |
| | mouth. | | | |
| CONJUNCTIVAL | Administration to the conjunctiva, | CONJUNC | 068 | C38194 |
| | the delicate membrane that lines | | | |
| | the eyelids and covers the exposed | | | |
| | surface of the eyeball. | | | |
| CUTANEOUS | Administration to the skin. | CUTAN | 130 | C38675 |
| DENTAL | Administration to a tooth or teeth. | DENTAL | 038 | C38197 |
| ELECTRO-OSMOSIS | Administration of through the | EL-OSMOS | 357 | C38633 |
| | diffusion of substance through a | | | |
| | membrane in an electric field. | | | |
| ENDOCERVICAL | Administration within the canal of | E-CERVIC | 131 | C38205 |
| | the cervix uteri. Synonymous with | | | |
| | the term intracervical.. | | | |
| ENDOSINUSIAL | Administration within the nasal | E-SINUS | 133 | C38206 |
| | sinuses of the head. | | | |
| ENDOTRACHEAL | Administration directly into the | E-TRACHE | 401 | C38208 |
| | trachea. | | | |
| ENTERAL | Administration directly into the | ENTER | 313 | C38209 |
| | intestines. | | | |
| EPIDURAL | Administration upon or over the | EPIDUR | 009 | C38210 |
| | dura mater. | | | |
| EXTRA-AMNIOTIC | Administration to the outside of the | X-AMNI | 402 | C38211 |
| | membrane enveloping the fetus | | | |
| EXTRACORPOREAL | Administration outside of the body. | X-CORPOR | 057 | C38212 |
| HEMODIALYSIS | Administration through | HEMO | 140 | C38200 |
| | hemodialysate fluid. | | | |
| INFILTRATION | Administration that results in | INFIL | 361 | C38215 |
| | substances passing into tissue | | | |
| | spaces or into cells. | | | |
| INTERSTITIAL | Administration to or in the | INTERSTIT | 088 | C38219 |
| | interstices of a tissue. | | | |
| INTRA-ABDOMINAL | Administration within the | I-ABDOM | 056 | C38220 |
| | abdomen. | | | |
| INTRA-AMNIOTIC | Administration within the amnion. | I-AMNI | 060 | C38221 |
| INTRA-ARTERIAL | Administration within an artery or | I-ARTER | 037 | C38222 |
| | arteries. | | | |
| INTRA-ARTICULAR | Administration within a joint. | I-ARTIC | 007 | C38223 |
| INTRABILIARY | Administration within the bile, bile | I-BILI | 362 | C38224 |
| | ducts or gallbladder. | | | |
| INTRABRONCHIAL | Administration within a bronchus. | I-BRONCHI | 067 | C38225 |
| INTRABURSAL | Administration within a bursa. | I-BURSAL | 025 | C38226 |
| INTRACARDIAC | Administration with the heart. | I-CARDI | 027 | C38227 |
| INTRACARTILAGINOUS | Administration within a cartilage; | I-CARTIL | 363 | C38228 |
| | endochondral. | | | |
| INTRACAUDAL | Administration within the cauda | I-CAUDAL | 413 | C38229 |
| | equina. | | | |
| INTRACAVERNOUS | Administration within a pathologic | I-CAVERN | 132 | C38230 |
| | cavity, such as occurs in the lung | | | |
| | in tuberculosis. | | | |
| INTRACAVITARY | Administration within a non- | I-CAVIT | 023 | C38231 |
| | pathologic cavity, such as that of | | | |
| | the cervix, uterus, or penis, or such | | | |
| | as that which is formed as the | | | |
| | result of a wound. | | | |
| INTRACEREBRAL | Administration within the | I-CERE | 404 | C38232 |
| | cerebrum. | | | |
| INTRACISTERNAL | Administration within the cisterna | I-CISTERN | 405 | C38233 |
| | magna cerebellomedularis. | | | |
| INTRACORNEAL | Administration within the cornea | I-CORNE | 406 | C38234 |
| | (the transparent structure forming | | | |
| | the anterior part of the fibrous tunic | | | |
| | of the eye). | | | |
| INTRACORONAL, | Administration of a drug within a | I-CORONAL | 117 | C38217 |
| DENTAL | portion of a tooth which is covered | | | |
| | by enamel and which is separated | | | |
| | from the roots by a slightly | | | |
| | constricted region known as the | | | |
| | neck. | | | |
| INTRACORONARY | Administration within the coronary | I-CORONARY | 119 | C38218 |
| | arteries. | | | |
| INTRACORPORUS | Administration within the dilatable | I-CORPOR | 403 | C38235 |
| CAVERNOSUM | spaces of the corporus cavernosa of | | | |
| | the penis. | | | |
| INTRADERMAL | Administration within the dermis. | I-DERMAL | 008 | C38238 |
| INTRADISCAL | Administration within a disc. | I-DISCAL | 121 | C38239 |
| INTRADUCTAL | Administration within the duct of a | I-DUCTAL | 123 | C38240 |
| | gland. | | | |
| INTRADUODENAL | Administration within the | I-DUOD | 047 | C38241 |
| | duodenum. | | | |
| INTRADURAL | Administration within or beneath | I-DURAL | 052 | C38242 |
| | the dura. | | | |
| INTRAEPIDERMAL | Administration within the | I-EPIDERM | 127 | C38243 |
| | epidermis. | | | |
| INTRAESOPHAGEAL | Administration within the | I-ESO | 072 | C38245 |
| | esophagus. | | | |
| INTRAGASTRIC | Administration within the stomach. | I-GASTRIC | 046 | C38246 |
| INTRAGINGIVAL | Administration within the gingivae. | I-GINGIV | 307 | C38247 |
| INTRAILEAL | Administration within the distal | I-ILE | 365 | C38249 |
| | portion of the small intestine, from | | | |
| | the jejunum to the cecum. | | | |
| INTRALESIONAL | Administration within or | I-LESION | 042 | C38250 |
| | introduced directly into a localized | | | |
| | lesion. | | | |
| INTRALUMINAL | Administration within the lumen of | I-LUMIN | 310 | C38251 |
| | a tube. | | | |
| INTRALYMPHATIC | Administration within the lymph. | I-LYMPHAT | 352 | C38252 |
| INTRAMEDULLARY | Administration within the marrow | I-MEDUL | 408 | C38253 |
| | cavity of a bone. | | | |
| INTRAMENINGEAL | Administration within the | I-MENIN | 409 | C38254 |
| | meninges (the three membranes | | | |
| | that envelope the brain and spinal | | | |
| | cord). | | | |
| INTRAMUSCULAR | Administration within a muscle. | IM | 005 | C28161 |
| INTRAOCULAR | Administration within the eye. | I-OCUL | 036 | C38255 |
| INTRAOVARIAN | Administration within the ovary. | I-OVAR | 354 | C38256 |
| INTRAPERICARDIAL | Administration within the | I-PERICARD | 314 | C38257 |
| | pericardium. | | | |
| INTRAPERITONEAL | Administration within the | I-PERITON | 004 | C38258 |
| | peritoneal cavity. | | | |
| INTRAPLEURAL | Administration within the pleura. | I-PLEURAL | 043 | C38259 |
| INTRAPROSTATIC | Administration within the prostate | I-PROSTAT | 061 | C38260 |
| | gland. | | | |
| INTRAPULMONARY | Administration within the lungs or | I-PULMON | 414 | C38261 |
| | its bronchi. | | | |
| INTRASINAL | Administration within the nasal or | I-SINAL | 010 | C38262 |
| | periorbital sinuses. | | | |
| INTRASPINAL | Administration within the vertebral | I-SPINAL | 022 | C38263 |
| | column. | | | |
| INTRASYNOVIAL | Administration within the synovial | I-SYNOV | 019 | C38264 |
| | cavity of a joint. | | | |
| INTRATENDINOUS | Administration within a tendon. | I-TENDIN | 049 | C38265 |
| INTRATESTICULAR | Administration within the testicle. | I-TESTIC | 110 | C38266 |
| INTRATHECAL | Administration within the | IT | 103 | C38267 |
| | cerebrospinal fluid at any level of | | | |
| | the cerebrospinal axis, including | | | |
| | injection into the cerebral | | | |
| | ventricles. | | | |
| INTRATHORACIC | Administration within the thorax | I-THORAC | 006 | C38207 |
| | (internal to the ribs); synonymous | | | |
| | with the term endothoracic. | | | |
| INTRATUBULAR | Administration within the tubules | I-TUBUL | 353 | C38268 |
| | of an organ. | | | |
| INTRATUMOR | Administration within a tumor. | I-TUMOR | 020 | C38269 |
| INTRATYMPANIC | Administration within the aurus | I-TYMPAN | 366 | C38270 |
| | media. | | | |
| INTRAUTERINE | Administration within the uterus. | I-UTER | 028 | C38272 |
| INTRAVASCULAR | Administration within a vessel or | I-VASC | 021 | C38273 |
| | vessels. | | | |
| INTRAVENOUS | Administration within or into a | IV | 002 | C38276 |
| | vein or veins. | | | |
| INTRAVENOUS BOLUS | Administration within or into a | IV BOLUS | 138 | C38274 |
| | vein or veins all at once. | | | |
| INTRAVENOUS DRIP | Administration within or into a | IV DRIP | 137 | C38279 |
| | vein or veins over a sustained | | | |
| | period of time. | | | |
| INTRAVENTRICULAR | Administration within a ventricle. | I-VENTRIC | 048 | C38277 |
| INTRAVESICAL | Administration within the bladder. | I-VESIC | 128 | C38278 |
| INTRAVITREAL | Administration within the vitreous | I-VITRE | 311 | C38280 |
| | body of the eye. | | | |
| IONTOPHORESIS | Administration by means of an | ION | 055 | C38203 |
| | electric current where ions of | | | |
| | soluble salts migrate into the | | | |
| | tissues of the body. | | | |
| IRRIGATION | Administration to bathe or flush | IRRIG | 032 | C38281 |
| | open wounds or body cavities. | | | |
| LARYNGEAL | Administration directly upon the | LARYN | 364 | C38282 |
| | larynx. | | | |
| NASAL | Administration to the nose; | NASAL | 014 | C38284 |
| | administered by way of the nose. | | | |
| NASOGASTRIC | Administration through the nose | NG | 071 | C38285 |
| | and into the stomach, usually by | | | |
| | means of a tube. | | | |
| NOT APPLICABLE | Routes of administration are not | NA | 312 | C48623 |
| | applicable. | | | |
| OCCLUSIVE DRESSING | Administration by the topical route | OCCLUS | 134 | C38286 |
| TECHNIQUE | which is then covered by a dressing | | | |
| | which occludes the area. | | | |
| OPHTHALMIC | Administration to the external eye. | OPHTHALM | 012 | C38287 |
| ORAL | Administration to or by way of the | ORAL | 001 | C38288 |
| | mouth. | | | |
| OROPHARYNGEAL | Administration directly to the | ORO | 410 | C38289 |
| | mouth and pharynx. | | | |
| OTHER | Administration is different from | OTHER | 135 | C38290 |
| | others on this list. | | | |
| PARENTERAL | Administration by injection, | PAREN | 411 | C38291 |
| | infusion, or implantation. | | | |
| PERCUTANEOUS | Administration through the skin. | PERCUT | 113 | C38676 |
| PERIARTICULAR | Administration around a joint. | P-ARTIC | 045 | C38292 |
| PERIDURAL | Administration to the outside of the | P-DURAL | 050 | C38677 |
| | dura mater of the spinal cord.. | | | |
| PERINEURAL | Administration surrounding a nerve | P-NEURAL | 412 | C38293 |
| | or nerves. | | | |
| PERIODONTAL | Administration around a tooth. | P-ODONT | 040 | C38294 |
| RECTAL | Administration to the rectum. | RECTAL | 016 | C38295 |
| RESPIRATORY | Administration within the | RESPIR | 136 | C38216 |
| (INHALATION) | respiratory tract by inhaling orally | | | |
| | or nasally for local or systemic | | | |
| | effect. | | | |
| RETROBULBAR | Administration behind the pons or | RETRO | 034 | C38296 |
| | behind the eyeball. | | | |
| SOFT TISSUE | Administration into any soft tissue. | SOFT TIS | 109 | C38198 |
| SUBARACHNOID | Administration beneath the | S-ARACH | 066 | C38297 |
| | arachnoid. | | | |
| SUBCONJUNCTIVAL | Administration beneath the | S-CONJUNC | 096 | C38298 |
| | conjunctiva. | | | |
| SUBCUTANEOUS | Administration beneath the skin; | SC | 003 | C38299 |
| | hypodermic. Synonymous with the | | | |
| | term SUBDERMAL. | | | |
| SUBLINGUAL | Administration beneath the tongue. | SL | 024 | C38300 |
| SUBMUCOSAL | Administration beneath the mucous | S-MUCOS | 053 | C38301 |
| | membrane. | | | |
| TOPICAL | Administration to a particular spot | TOPIC | 011 | C38304 |
| | on the outer surface of the | | | |
| | body. The E2B term | | | |
| | TRANSMAMMARY is a subset of | | | |
| | the term TOPICAL. | | | |
| TRANSDERMAL | Administration through the dermal | T-DERMAL | 358 | C38305 |
| | layer of the skin to the systemic | | | |
| | circulation by diffusion. | | | |
| TRANSMUCOSAL | Administration across the mucosa. | T-MUCOS | 122 | C38283 |
| TRANSPLACENTAL | Administration through or across | T-PLACENT | 415 | C38307 |
| | the placenta. | | | |
| TRANSTRACHEAL | Administration through the wall of | T-TRACHE | 355 | C38308 |
| | the trachea. | | | |
| TRANSTYMPANIC | Administration across or through | T-TYMPAN | 124 | C38309 |
| | the tympanic cavity. | | | |
| UNASSIGNED | Route of administration has not yet | UNAS | 400 | C38310 |
| | been assigned. | | | |
| UNKNOWN | Route of administration is | UNKNOWN | 139 | C38311 |
| | unknown. | | | |
| URETERAL | Administration into the ureter. | URETER | 112 | C38312 |
| URETHRAL | Administration into the urethra. | URETH | 017 | C38271 |
| VAGINAL | Administration into the vagina. | VAGIN | 015 | C38313 |

In some embodiments, the route of administration may be oral, intranasal, inhalation, intravenous, topical, injectable and/or transdermal. Topical and transdermal include application to the face or head, and injectable would include subcutaneous injection to the face or head, or injection proximal to, or distal to, the trigeminal nerve. Injection distal to the trigeminal nerve may be proximal to a site of pain that is distal to the trigeminal nerve.

The formulator will understand that excipients are used primarily to serve in delivering a safe, stable, and functional pharmaceutical, serving not only as part of the overall vehicle for delivery but also as a means for achieving effective absorption by the recipient of the active ingredient. An excipient may fill a role as simple and direct as being an inert filler, or an excipient as used herein may be part of a pH stabilizing system or coating.

Pharmaceutical compositions may comprise one or more pharmaceutically acceptable carriers, excipients, or diluents. Examples of such carriers are well known to those skilled in the art and can be prepared in accordance with acceptable pharmaceutical procedures, such as, for example, those described in Remington's Pharmaceutical Sciences, 17th edition, ed. Alfonoso R. Gennaro, Mack Publishing Company, Easton, Pa. (1985), the entire disclosure of which is incorporated by reference herein for all purposes. As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the formulation and are biologically acceptable. Supplementary active ingredients can also be incorporated into the pharmaceutical compositions.

Compounds of the present teachings can be administered orally or parenterally, neat or in combination with conventional pharmaceutical carriers. Applicable solid carriers can include one or more substances which can also act as flavoring agents, lubricants, solubilizers, suspending agents, fillers, glidants, compression aids, binders or tablet-disintegrating agents, or encapsulating materials. Oral formulations containing a compound disclosed herein can comprise any conventionally used oral form, including tablets, capsules, buccal forms, troches, lozenges and oral liquids, suspensions or solutions. In powders, the carrier can be a finely divided solid, which is an admixture with a finely divided compound. In tablets, a compound disclosed herein can be mixed with a carrier having the necessary compression properties in suitable proportions and compacted in the shape and size desired. The powders and tablets can contain up to 99% of the compound.

Capsules can contain mixtures of one or more compound(s) disclosed herein with inert filler(s) and/or diluent(s) such as pharmaceutically acceptable starches (e.g., corn, potato or tapioca starch), sugars, artificial sweetening agents, powdered celluloses (e.g., crystalline and microcrystalline celluloses), flours, gelatins, gums, and the like.

Useful tablet formulations can be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, ion exchange resins, benzyl alcohol, eucalyptol, gelatin, limonene, mannitol, menthol, menthone, menthyl acetate, sucralose, and vanillin. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine. Oral formulations herein can utilize standard delay or time-release formulations to alter the absorption of the compound(s). The oral formulation can also consist of administering a compound disclosed herein in water or fruit juice, containing appropriate solubilizers or emulsifiers as needed.

Liquid carriers can be used in preparing solutions for oral or parenteral administration (such as intravenous, intramuscular, or other injections), including suspensions, emulsions, syrups, elixirs, and additionally for inhaled delivery. A compound of the present teachings can be dissolved or suspended in a pharmaceutically acceptable liquid carrier such as water, an organic solvent, or a mixture of both, or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, and osmo-regulators. Examples of liquid carriers for oral and parenteral administration include, but are not limited to, water (particularly containing additives as described herein, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example, intramuscular, intraperitoneal or subcutaneous injection. Sterile injectable solutions can also be administered intravenously. Compositions for oral administration can be in either liquid or solid form.

Preferably the pharmaceutical composition is in unit dosage form, for example, as tablets, capsules, powders, solutions, suspensions, emulsions, granules, or suppositories. In such form, the pharmaceutical composition can be further sub-divided to contain appropriate quantities of the compound. The unit dosage forms can be packaged compositions, for example, packeted powders, vials, ampoules, prefilled syringes or sachets containing liquids. Alternatively, the unit dosage form can be a capsule or tablet itself, or it can be the appropriate number of any such compositions in package form. Such doses can be administered in any manner useful in directing the compound(s) to the recipient's bloodstream, including orally, via implants, parenterally (including intravenous, intraperitoneal and subcutaneous injections), rectally, vaginally, and transdermally.

When administered for the treatment or inhibition of a particular disease state or disorder, it is understood that an effective dosage can vary depending upon the particular compound utilized, the pharmaceutical composition formulated, the mode of administration, and severity of the condition being treated, as well as the various physical factors related to the individual being treated. In therapeutic applications, a compound of the present teachings can be provided to a patient already suffering from a disease in an amount sufficient to cure or at least partially ameliorate the symptoms of the disease and its complications. The dosage to be used in the treatment of a specific individual typically must be subjectively determined by the attending physician. The variables involved include the specific condition and its state as well as the size, age and response pattern of the patient.

In some cases it may be desirable to administer a compound directly to the airways of the patient, using devices such as, but not limited to, metered dose inhalers, breath-operated inhalers, multidose dry-powder inhalers, pumps, squeeze-actuated nebulized spray dispensers, aerosol dispensers, and aerosol nebulizers. For administration by intranasal or intrabronchial inhalation, the compounds of the present teachings can be formulated into a liquid composition, a solid composition, or an aerosol composition. The liquid composition can include, by way of illustration, one or more compounds of the present teachings dissolved, partially dissolved, or suspended in one or more pharmaceutically acceptable solvents and can be administered by, for example, a pump or a squeeze-actuated nebulized spray dispenser. The solvents can be, for example, isotonic saline or bacteriostatic water. The solid composition can be, by way of illustration, a powder preparation including one or more compounds of the present teachings intermixed with lactose or other inert powders that are acceptable for intrabronchial use, and can be administered by, for example, an aerosol dispenser or a device that breaks or punctures a capsule encasing the solid composition and delivers the solid composition for inhalation. The aerosol composition can include, by way of illustration, one or more compounds of the present teachings, propellants, surfactants, and co-solvents, and can be administered by, for example, a metered device. The propellants can be a chlorofluorocarbon (CFC), a hydrofluoroalkane (HFA), or other propellants that are physiologically and environmentally acceptable.

Compounds described herein can be administered parenterally or intraperitoneally. Solutions or suspensions of these compounds or a pharmaceutically acceptable salts, hydrates, or esters thereof can be prepared in water suitably mixed with a surfactant such as hydroxyl-propylcellulose. Dispersions can also be prepared in glycerol, liquid polyethylene glycols, and mixtures thereof in oils. Under ordinary conditions of storage and use, these preparations typically contain a preservative to inhibit the growth of microorganisms.

The pharmaceutical forms suitable for injection can include sterile aqueous solutions or dispersions and sterile powders for the extemporaneous preparation of sterile injectable solutions or dispersions. In some embodiments, the form can sterile and its viscosity permits it to flow through a syringe. The form preferably is stable under the conditions of manufacture and storage and can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Compounds described herein can be administered transdermally, i.e., administered across the surface of the body and the inner linings of bodily passages including epithelial and mucosal tissues. Such administration can be carried out using the compounds of the present teachings including pharmaceutically acceptable salts, hydrates, or esters thereof, in lotions, creams, foams, patches, suspensions, solutions, and suppositories (rectal and vaginal).

Transdermal administration can be accomplished through the use of a transdermal patch containing a compound, such as a compound disclosed herein, and a carrier that can be inert to the compound, can be non-toxic to the skin, and can allow delivery of the compound for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the compound can also be suitable. A variety of occlusive devices can be used to release the compound into the blood stream, such as a semi-permeable membrane covering a reservoir containing the compound with or without a carrier, or a matrix containing the compound. Other occlusive devices are known in the literature. Compounds described herein can be administered rectally or vaginally in the form of a conventional suppository. Suppository formulations can be made from traditional materials, including cocoa butter, with or without the addition of waxes to alter the suppository's melting point, and glycerin. Water-soluble suppository bases, such as polyethylene glycols of various molecular weights, can also be used.

Lipid formulations or nanocapsules can be used to introduce compounds of the present teachings into host cells either in vitro or in vivo. Lipid formulations and nanocapsules can be prepared by methods known in the art.

Inhibitors at the receptor level to CGRP are postulated to be useful in pathophysiologic conditions where excessive CGRP receptor activation has occurred. Such excessive CGRP receptor activation may occur in trigeminal neuralgia.

Another aspect of the disclosure is a method of inhibiting the CGRP receptor comprising contacting the CGRP receptor with a compound of formula I, formula II, formula III, or a pharmaceutically acceptable salt(s) thereof.

Another aspect of the disclosure is a method for treating conditions associated with aberrant levels of CGRP comprising the administration of a therapeutically effective amount of a compound of formula I, formula II, and/or formula III to a patient.

Another aspect of the disclosure is the use of a compound of formula I, formula II, and/or formula III in the manufacture of a medicament for the treatment of conditions related to aberrant levels of CGRP, such as trigeminal neuralgia. Another aspect of the disclosure is a compound according to formula I, formula II, and/or formula III useful for the treatment of conditions related to aberrant levels of CGRP, such as trigeminal neuralgia. Another aspect of the disclosure is a method of treating trigeminal neuralgia. Another aspect of the disclosure relates to a method of treating inflammation (particularly neurogenic inflammation), pain, and other conditions or symptoms related to trigeminal neuralgia, the treatment of which can be effected by the antagonism of the CGRP receptor by the administration of pharmaceutical compositions comprising one or more compounds of Formula I, Formula II, and/or Formula III as defined herein.

Another aspect of the disclosure relates to a method of treatment using combinations of one or more of Formulas I, Formula II, and/or Formula III with one or more therapeutic agents selected from phenytoin (dilantin), gabapentin (Neurontin), Lamotrigine (Lamictal), Oxcarbazepine (Trileptal), Topiramate (Topamax), baclofen (Lioresal), carbamazepine, Tegretol, Epitol, Carbatrol, clonazepam, Botox injection, glycerol injection (rhizotomy), or any other known therapeutic agent effective against trigeminal neuralgia. Also contemplated are methods of treatment further comprising one or more pain relievers such as ibuprofen, acetaminophen, naproxen sodium, diclofenac, corticosteroids, opioids, antidepressants, anticonvulsants, NSAIDs, lidocaine, and other known pain treatments.

Another aspect of the disclosure relates to a method of treatment using combinations of one or more compounds of Formulas I, Formula II, and/or Formula III in a patient having undergone one or more surgical procedures for relieving trigeminal neuralgia. In patients having undergone one or more surgical procedures for relieving trigeminal neuralgia, the compounds and compositions of the present disclosure may treat or prevent residual pain and symptoms of trigeminal neuralgia, or may prevent flares or recurrence of trigeminal neuralgia. The surgical procedures for relieving trigeminal neuralgia may be one or more of microvascular decompression, brain stereotactic radiosurgery (Gamma knife), rhizotomy, glycerol injection (rhizotomy), balloon compression (rhizotomy), radiofrequency thermal lesioning (rhizotomy), or any other surgical technique known to treat trigeminal neuralgia.

### Pharmacokinetics and Dosing

In some embodiments, a method of treating trigeminal neuralgia may be characterized by one or more pharmacokinetic parameters such as AUC, Cₘₐₓ, Tₘₐₓ, and others known and understood to persons of skill in the art. The term “pharmacokinetic” (PK) as used herein is used in its ordinary sense to mean the pharmacokinetic aspects of drug delivery. By definition pharmacokinetics (PK) is the study of how an organism affect a drug, e.g. how and how fast it metabolizes the drug. The pharmacokinetics typically vary based upon the dosage amount of one or more compounds of Formula I, Formula II, and/or Formula III The pharmacokinetics may or may not vary as a function of administration route.

In some embodiments, a method of treating trigeminal neuralgia may be characterized by an AUC for a compound according to Formula I, Formula II, and/or Formula III. In some embodiments, the AUC₀₋ₜ and/or AUC_{0-inf} (collectively referred to in the alternative as, simply, AUC) may be from about 80-125% of a given AUC value. In some embodiments, the AUC may be within 80-125% of about 25, 50, 100, 150, 200, 250, 300, 400, 500, 600, 700, 800, 900, 1000, 1300, 1600, 1900, 2500, 3500, 5000, 7500, 10,000, 15,000, 20,000, 30,000, 50,000, and/or 100,000 hr*ng/mL. In some embodiments, a systemic treatment may have a larger AUC than a localized (such as topical or subdermal) treatment.

In some embodiments, a method of treating trigeminal neuralgia may be characterized by a Cₘₐₓ for a compound according to Formula I, Formula II, and/or Formula III. In some embodiments, the Cₘₐₓ may be from about 80-125% of a given Cₘₐₓ value. In some embodiments, the Cₘₐₓ may be within 80-125% of about 1, 2, 3, 4, 5, 8, 10, 20, 35, 50, 80, 100, 150, 200, 250, 300, 400, 500, 600, 700, 800, 900, 1000, 1300, 1600, 1900, 2500, 3500, 5000, 7500, 10,000, 15,000, 20,000, 30,000, 50,000, and/or 100,000 ng/mL. In some embodiments, a systemic treatment may have a larger Cₘₐₓ than a localized (such as topical or subdermal) treatment.

In some embodiments, a ratio Cₘₐₓ/AUC may be used to characterize a method of treating trigeminal neuralgia wherein one or more of a compound according to Formula I, Formula II, and/or Formula III are administered to a subject. In some embodiments, the Cₘₐₓ/AUC ratio may be from about 80-125% of a given Cₘₐₓ/AUC ratio. In some embodiments, the Cₘₐₓ/AUC ratio may be from about 80-125% of about 0.01, 0.03, 0.05, 0.08, 0.1, 0.13, 0.17, 0.2, 0.25, 0.3, 0.35, 0.4, 0.45, 0.5, 0.6, 0.7, 0.8, and 0.9.

In some embodiments, the Tₘₐₓ may range from about 0.1-16 hours, or from about 0.3-8 hours, or from about 0.5-4 hours, or from 0.5-2 hours, or from about 1-2 hours. In some embodiments, the route of administration, which may be any route described herein or known to a person of skill in the art, may affect the Tₘₐₓ of a compound according to Formula I, Formula II, and/or Formula III. Excipients or other pharmaceutically acceptable carriers in pharmaceutical compositions of one or more compounds according to Formula I, Formula II, and/or Formula III may alter the Tₘₐₓ value by making it larger or smaller than a pharmaceutical composition having the excipient or carrier excluded.

Solid compositions are normally formulated in dosage units providing from about 0.1 to about 1000 mg of the active ingredient per dose. Some examples of solid dosage units are 0.1 mg, 1 mg, 10 mg, 100 mg, 500 mg, and 1000 mg. Liquid compositions are generally in a unit dosage range of 0.1-100 mg/mL. Some examples of liquid dosage units are 0.1 mg/mL, 1 mg/mL, 10 mg/mL, 25 mg/mL, 50 mg/mL, and 100 mg/mL.

In some embodiments, a dose is daily. In some embodiments, a dose is twice daily. In some embodiments, a does is one, two, three, four, or five times daily. In some embodiments, a dose is once every other day, once every second day, once every third day, once every fourth day, once every fifth day, once every sixth day, weekly, bi-weekly, or monthly. Typically, a dose may be from 0.01-100 mg/kg body weight, or from 0.05-50 mg/kg body weight, or from 0.1-10 mg/kg body weight, or from 0.15-5 mg/kg body weight, or from 0.2-2 mg/kg body weight, or from 0.5-1.5 mg/kg body weight, or from 1-1.5 mg/kg body weight. In some embodiments, the dosing regimen may comprise one or more optional loading doses and a subsequent maintenance dose regimen. A loading dose may be larger than the doses given in a subsequent maintenance dose regimen.

In some embodiments, the dosage is adjusted based upon trigeminal neuralgia symptoms observed in the patient. A symptom-dependent regimen may have a larger treatment dose while symptoms are present and a smaller preventative dose while symptoms are lessened, in remission, controlled, etc. In some embodiments, a treatment dose may comprise a unit dosage of about 10 mg, 20 mg, 30 mg, 40 mg, 50 mg, 60 mg, 70 mg, 75 mg, 80 mg, 100 mg, 120 mg, 140 mg, 160 mg, 180 mg, or 200 mg of a CGRP receptor antagonist compound according to one or more of Formula I, Formula II, and/or Formula III. In some embodiments, a preventative dose may comprise a unit dosage of about 1 mg, 2 mg, 5 mg, 10 mg, 20 mg, 30 mg, 40 mg, 50 mg, 60 mg, 70 mg, 75 mg, 80 mg, 100 mg, 120 mg, or 140 mg of a CGRP receptor antagonist compound according to one or more of Formula I, Formula II, and/or Formula III. In some embodiments, the preventative dose is an amount that is about ⅞, ¾, ⅝, ½, ⅜, ¼, or about ⅛ of the amount of the treatment dose. In some embodiments, if a patient receiving a preventative dosing regimen experiences recurrence or onset of symptoms, a treatment dosing regimen may replace the preventative dosing regimen until symptoms have lessened or subsided. Each of the treatment and/or preventative doses may be adjusted during the regimen based upon the severity of trigeminal neuralgia symptoms and changes in the symptoms.

### Definitions

“Therapeutically effective” means there is a meaningful patient benefit as understood by medical practitioners.

“Patient” means a person who may benefit from treatment as determined by medical practitioners.

“Neuralgia” as used herein refers to pain caused by irritated or damaged nerves occurring in any part of the body. Generally, it is contemplated that the CGRP receptor antagonists of Formulas (I), (II), and (III) are useful in methods of treatment of neuralgia, including specific neuralgias such as trigeminal neuralgia. “Neuralgia” is also intended to be fully encompassing of neuropathy, neuropathic pain, and general nerve pain or irritation.

“Trigeminal Neuralgia” as used herein includes the description in the International Classification of Diseases, 11^{th} Revision (ICD-11)—“Trigeminal neuralgia is a manifestation of orofacial neuropathic pain restricted to one or more divisions of the trigeminal nerve. The pain is recurrent, abrupt in onset and termination, triggered by innocuous stimuli and typically compared to an electric shock or described as shooting or stabbing. Some patients experience continuous pain between these painful paroxysms.” The term “Trigeminal Neuralgia” more generally refers to any pain associated with or due to the trigeminal nerve. Trigeminal neuralgia may be classical, or caused by intracranial vascular compression of the trigeminal nerve root, secondary, or as a result of another disease or condition such as multiple sclerosis or a tumor, or idiopathic, in which no apparent cause of nerve disturbance is identified. Trigeminal neuralgia may also be atypical, characterized by persistent pain, or pain lasting longer than about 2 minutes. The bouts of pain associated with classical trigeminal neuralgia typically last from about a fraction of a second to about 2 minutes. The term also includes pain or symptoms secondary to trigeminal neuralgia, pain or symptoms secondary to or as a result of a surgical procedure to treat or prevent trigeminal neuralgia, or any other pain, symptom, or condition secondary to trigeminal neuralgia. Trigeminal neuralgia and symptoms thereof may be episodic, sporadic, triggered, in delayed response to a trigger, chronic, continuous, etc. and treatment and/or prevention for each are contemplated. Further symptoms of trigeminal neuralgia may also include subtle facial movements, including blinks or mouth movements, and mild hypoesthesia. Forceful contraction of the facial muscles “tic convulsive” may also be a symptom. The term “trigeminal neuralgia” as used herein is not intended to strictly limit the scope of the treatment methods disclosed herein—if a method disclosed for treating “trigeminal neuralgia” is more broadly applicable to neuralgia, it can be appreciated that treatment of any neuralgia is also contemplated.

“Trigeminal neuralgia” may be generally characterized under two different types: type 1 (TN1) and type 2 (TN2). TN1 (also known as classic is generally characterized by sporadic or periodic attacks of stabbing, intense pain. TN2 is generally characterized by constant aching or burning of a lower intensity than TN1. A patient may present with either of, or both of, TN1 and TN2. In some embodiments, provided for are methods of treating TN1 with one or more of a compound according to Formula I. In some embodiments, provided for are methods of treating TN1 with one or more of a compound according to Formula II. In some embodiments, provided for are methods of treating TN1 with one or more of a compound according to Formula III. In some embodiments, provided for are methods of treating TN2 with one or more of a compound according to Formula I. In some embodiments, provided for are methods of treating TN2 with one or more of a compound according to Formula II. In some embodiments, provided for are methods of treating TN2 with one or more of a compound according to Formula III. In some embodiments, provided for are methods of treating both TN1 and TN2 with one or more of a compound according to Formula I. In some embodiments, provided for are methods of treating both TN1 and TN2 with one or more of a compound according to Formula II. In some embodiments, provided for are methods of treating both TN1 and TN2 with one or more of a compound according to Formula III.

“CGRP Receptor Antagonist” as used herein generally may refer to any small molecule antagonist of the CGRP receptor that is useful for treating, ameliorating, and/or preventing a disease or indication. In some embodiments, a CGRP receptor antagonist may be given as a pharmaceutical composition. In some embodiments, one or more CGRP receptor antagonists may be included in a pharmaceutical composition.

The terms “treating” and its derivatives such as “treat” or “treatment,” as used herein, may be used with respect to a particular condition, for example, a condition due to or associated with CGRP and/or abberant levels thereof, such as neuralgia, including trigeminal neuralgia. In reference to a particular condition, “treating” and its derivatives are inclusive of several meanings, including (1) to alleviate one or more symptoms, effects, or side effects associated with the condition, (2) to ameliorate the condition and/or one or more of the biological manifestations or underlying causes of the condition, (3) to interfere with one or more of the biological manifestations or underlying causes of the condition or with one or more points in the biological cascade(s) associated with the condition, (4) to slow the progression of, or arrest the development of, the condition or of one or more of the biological manifestations of the condition, (5) to prevent or reduce the risk of a subject developing the condition, in some cases prophylactically when the subject has one or more risk factors for the condition (6) to cause regression of the condition, or improvement or reversal of, the biological manifestations or underlying causes of the conditions. It can be appreciated that “treating” may encompass one or more of these meanings simultaneously and that a subject's condition may change over time or throughout the course of treatment such that the meaning of “treating” as applied to a given subject may change over time or throughout the course of treatment. “Treating” as used herein may also refer to any beneficial effect of administering a CGRP receptor antagonist compound and/or a pharmaceutical composition comprising a CGRP receptor antagonist to a subject or patient having or being at risk for developing trigeminal neuralgia. “Treating” therefore may encompass alleviating trigeminal neuralgia and/or the symptoms of trigeminal neuralgia, prophylaxis or prevention of trigeminal neuralgia or of the worsening of trigeminal neuralgia, and reducing the risk that a subject or patient may develop new or worsening trigeminal neuralgia. “Treatment” may be in combination with other therapies or alone.

One or more treatment outcomes may be associated with “treatment” of trigeminal neuralgia. One such outcome is an improvement on the Numeric Pain Rating Scale (NPRS), which is a 0-10 scale of pain severity where 0 is no pain, 5 is moderate pain, and 10 is the worst possible pain. In an embodiment, the treatment results in an improvement of at least about 2 points in the NPRS. In alternative embodiments, the treatment results in an improvement of at least about 1 point in the NPRS, or at least about 3, 4, 5, 6, 7, 8, 9, or 10 points in the NPRS. Other outcomes include the Penn Facial Pain Scale, the Brief Pain Interference Index, the Penn Facial Pain Scale-Revised, the Pain Disability Index, the Patient Flobal Impression of Change Scale, or any other appropriate scale or method of assessing improvement in trigeminal neuralgia symptoms as a result of the treatment methods herein. Generally, an improvement (reduction in pain) under one or more outcomes may be indicate an effective treatment as would be understood by a person of skill in the art.

The term “AUC” (area under the curve) typically refers to a total amount of drug absorbed or exposed to a subject. Generally, AUC may be obtained from mathematical method in a plot of drug concentration in the subject over time until the concentration is negligible. The term “AUC” may also refer to partial AUC at specified time intervals. The AUC may be determined within a certain time period (such as from time 0 to time t; AUC₀₋ₜ) or may be extrapolated from the last measured point to the point where drug concentration in the subject is zero (AUC_{inf}).

The term “Cₘₐₓ” refers to a maximum concentration of a drug in blood, serum, a specified compartment or test area of a subject after administration of a dose or between administration of a first dose and administration of a second dose. The term Cₘₐₓ could also refer to dose normalized ratios, if specified.

The term “Tₘₐₓ” refers to a time or period after administration of a drug when the maximum concentration (Cₘₐₓ) is reached in blood, serum, a specified compartment or test area of a subject.

The term “dosing interval” refers to the amount of time that elapses between multiple doses of a formulation disclosed herein being administered to a subject. Dosing interval can thus be indicated as ranges.

The term “dosing frequency” refers to the frequency of administering doses of a formulation disclosed herein in a given time. Dosing frequency can be indicated as the number of doses per a given time, e.g., once a day (daily), twice a day (twice daily), once a week (weekly) or once in two weeks, etc.

For the purposes of the present disclosure, the term “excipient” and “carrier” are used interchangeably throughout the disclosure and said terms are defined herein as, “ingredients which are used in the practice of formulating a safe and effective pharmaceutical composition.”

### EXAMPLES

The following examples further describe and demonstrate embodiments within the scope of the present disclosure. The Examples are given solely for purpose of illustration and are not to be construed as limitations of the present disclosure, as many variations thereof are possible without departing from the spirit and scope of the disclosure.

### Example 1—Case Study—Rimegepant Oral Treatment for Trigeminal Neuralgia

A case study was performed in which trigeminal neuralgia was successfully treated for the first time with a small molecule CGRP receptor antagonist.

### Key Points:

Atypical trigeminal neuralgia is difficult to treat and many patients have refractory pain
Rimegepant is a small molecule CGRP antagonist that was used to successfully treat refractory atypical trigeminal neuralgia
The medication is well tolerated and maintains efficacy over a period of six months

### Abstract of Study:

In this study, refractory atypical trigeminal neuralgia was successfully treated using rimegepant, a novel oral calcitonin gene-related peptide (CGRP) antagonist. The patient experienced a decrease in pain from seven to nine out of 10 to 0 out of 10 within one day of therapy initiation. He has now been receiving the medication for over six months, and the treatment continues to be efficacious and well-tolerated with no significant adverse effects. Thus, this case illustrates the promise of CGRP antagonist therapy as a novel treatment for trigeminal neuralgia, including atypical trigeminal neuralgia.

### Case:

A 56-year-old man with a 5-year history of TN presented with episodic sharp, stabbing and constant burning pain along the V2 nerve distribution. His pain was triggered by sensation on his upper right teeth, including wind, mastication, and extreme-temperature fluids. His self-reported pain score ranged from seven to nine out of 10, and his Barrow Neurological Institute (BNI) pain intensity score was rated IV out of V, defined as inadequate pain control with medication. At presentation, his pain regimen consisted of 300 mg oxcarbazepine per day, up to eight 15 mg tablets of oxycodone per day, and 10 mcg per hour transdermal buprenorphine patch. On neurological exam, sensation was intact to light touch bilaterally except along the right nasolabial fold, and grimace was symmetric. The patient experienced impairment in ability to perform daily activities, such as eating, working, and speaking. Other associated symptoms included headache, fatigue, and significant weight loss.

Prior to presentation, the patient unsuccessfully tried several medications for pain control, including oxcarbazepine, meperidine, carisoprodol, propranolol, pregabalin, sumatriptan, and ergot. He also failed medical cannabinoids, oxygen therapy, trigger point injections, and sphenopalatine nerve blockades. He underwent four surgical procedures at outside institutions, including Gamma Knife radiosurgery (37 months prior to presentation), right balloon rhizotomy (31 months prior to presentation), radiofrequency ablation (22 months prior to presentation), and a V2-3 radiofrequency rhizotomy (14 months prior to presentation). Notably, after the radiofrequency rhizotomy, his sharp, stabbing pain episodes became more frequent, occurring daily, and the constant burning pain between attacks greatly increased in severity.

Magnetic resonance imaging demonstrated enhancement of V2, suggesting injury secondary to rhizotomy. Given his sharp/stabbing pain, we offered him a microvascular decompression (MVD). While the MVD successfully addressed the sharp, stabbing pain and dramatically reduced the frequency and severity of his attacks, he continued to experience the constant, burning atypical TN pain exacerbated by the prior rhizotomy.

After multiple trials of different medications for six years after his MVD, the patient was started on 75 mg of rimegepant per day. Within 12 hours of his first dose, his self-reported pain score improved to 0 out of 10. In his six-month treatment period, he experienced no significant adverse effects and mostly had no pain. He rarely had mild (2 out of 10) pain, which was controlled with 1 mg buprenorphine. His BNI pain intensity score was reduced to II, defined as no pain on medication. He also reported a drastic improvement in mood and cognition, associated with more meaningful connections in his personal and professional life, and was weaned from several of his pain medications.

### Discussion:

Given these initial findings that CGRP is implicated in trigeminal neuralgia and promising results describing the efficacy of CGRP inhibition in other craniofacial pain syndromes, clinical trials are being initiated to evaluate drugs targeting the CGRP pathway for trigeminal neuralgia (See Example 2 below). However, there is currently limited data regarding CGRP blockade as a therapy for trigeminal neuralgia.

Rimegepant, an orally-administered small molecule CGRP antagonist has proven efficacy in the preventive and acute treatment of migraine with a favorable safety profile. (Lipton R B, Croop R, Stock E G, et al. Rimegepant, an Oral Calcitonin Gene-Related Peptide Receptor Antagonist, for Migraine. N Eng/J Med. Published online Jul. 10, 2019; Croop R, Goadsby P J, Stock D A, et al. Efficacy, safety, and tolerability of rimegepant orally disintegrating tablet for the acute treatment of migraine: a randomised, phase 3, double-blind, placebo-controlled trial. The Lancet. 2019; 394(10200):737-745; Croop R, Lipton R B, Kudrow D, et al. Oral rimegepant for preventive treatment of migraine: a phase ⅔, randomised, double-blind, placebo-controlled trial. The Lancet. 2021; 397(10268):51-60. doi:10.1016/S0140-6736(20)32544-7). In the randomized acute treatment trial with the fast-dissolve orally disintegrating tablet (ODT) formulation, statistically significant benefit on achieving pain freedom was demonstrated at 90 minutes postdose and for pain relief at 60 minutes postdose. (Croop R, Goadsby P J et al.)

As seen in this case, the medication was similarly effective in the treatment of highly refractory trigeminal neuralgia with atypical components, with the patient experiencing rapid and significant improvement of his pain with no significant adverse effects. Given that carbamazepine and oxcarbazepine are the only oral therapies with a proven treatment benefit, the drastic, immediate, and sustained improvement in pain seen for this patient is promising.

While research on other anti-CGRP medications for trigeminal neuralgia is sparse, similarly positive results have been seen in one case series investigating erenumab, an injected anti-CGRP monoclonal antibody. This study demonstrated a significant decrease in trigeminal neuralgia pain in nine out of 10 patients treated with erenumab, with five patients reporting complete pain resolution. (Parascandolo E. et al) Notably, many of these patients also had trigeminal neuralgia that was refractory to multiple medications. Side effects were minimal, with three patients reporting constipation and/or injection site reaction

### Conclusion:

Trigeminal Neuralgia is a condition that carries significant morbidity and current treatments provide limited benefit for a significant number of patients with atypical trigeminal neuralgia. Thus, the significant treatment response and lack of adverse effects with rigemepant described in this case suggests that CGRP inhibition with this medication is a promising medical therapy for refractory atypical trigeminal neuralgia. This study also suggests that small molecule CGRP receptor antagonists, such as those according to Formulas (I), (II), and/or (Ill) herein, are useful for the treatment of trigeminal neuralgia based upon the surprising discovery that rimegepant is an effective treatment for trigeminal neuralgia.

### Example 2—Clinical Trial—Rimegepant Oral Treatment for Trigeminal Neuralgia

A clinical trial to evaluate the efficacy of a CGRP receptor antagonist (Rimegepant) for treating trigeminal neuralgia is described herein. The outlined clinical trial is generally applicable to and useful for any CGRP receptor antagonist described herein according to one or more of formulas (I), (II), and (Ill).

### Rationale:

Trigeminal Neuralgia is a neuropathic pain disorder characterized by recurrent, paroxysmal, lancinating pain in the distribution of one or more branches of the trigeminal nerve. These episodic bouts of severe facial pain can last seconds to minutes, occur several times per day, and often result in significant disability. Although the exact mechanism of action remains unclear, antiepileptic drugs, most notably carbamazepine and oxcarbazepine, remain the first-line treatment for these patients. Nonetheless, the majority of patients have an incomplete response to current pharmacotherapy and resort to more invasive procedures, including rhizotomy and surgical decompression of the microvasculature surrounding the trigeminal nerve. While numerous treatment options exist, inadequate relief of pain and recurrence of symptoms are common, prompting the need for novel pharmacologic interventions. (Montano N, Conforti G, Di Bonaventura R, Meglio M, Fernandez E, Papacci F. Advances in diagnosis and treatment of trigeminal neuralgia. Ther Clin Risk Manag 2015; 11:289-99)

Although the underlying mechanisms of classical Trigeminal Neuralgia have not been fully elucidated, imaging studies and surgical procedures clearly implicate vascular compression of the trigeminal nerve in the pathophysiology of the disease. (Goodwin C R, Yang J X, Bettegowda C, et al. Glycerol rhizotomy via a retrosigmoid approach as an alternative treatment for trigeminal neuralgia. Clin Neurol Neurosurg 2013; 115:2454-6). As a result of this nerve compression, subsequent axonal demyelination of the trigeminal nerve results in hyperexcitation and abnormal discharge of primary sensory neurons, ultimately contributing to the aberrant nociceptive transmission and neuropathic pain associated with this disorder. (Goodwin et al.) The neuropeptide Calcitonin Gene-Related Peptide (CGRP) and its aberrant release from the trigeminal nerve has been clearly implicated in the pathophysiology of migraine headache. (lyengar S, Ossipov M H, Johnson K W. The role of calcitonin gene-related peptide in peripheral and central pain mechanisms including migraine. Pain 2017; 158:543-59) In addition, CGRP is thought to play an important role in the development of neuronal sensitization and neuropathic pain (lyengar S et al.) and is a major mediator of pathologic vasodilatation of intracranial arteries. (Lassen L H, Haderslev P A, Jacobsen V B, Iversen H K, Sperling B, Olesen J. CGRP may play a causative role in migraine. Cephalalgia 2002; 22:54-61) Clinical studies have also shown increased levels of CGRP in the cerebrospinal fluid of patients with Trigeminal Neuralgia. (Qin Z L, Yang L Q, Li N, et al. Clinical study of cerebrospinal fluid neuropeptides in patients with primary trigeminal neuralgia. Clin Neurol Neurosurg 2016; 143:111-5.)

The study is based on the evolving preclinical and clinical evidence suggesting a role for CGRP in the development of neuropathic pain syndromes and Trigeminal Neuralgia. BHV-3000 (rimegepant) is a small molecule CGRP receptor antagonist for the treatment of Trigeminal Neuralgia. The data from this study assesses the safety and efficacy of rimegepant vs placebo in the treatment of Trigeminal Neuralgia.

### Target Population:

The study includes male and female patients 18 years of age and older with a clinical diagnosis of classical Trigeminal Neuralgia based on the International Classification of Headache Disorders, 3rd edition, who have had an inadequate response to current treatments and symptoms for at least three months. Additionally, patients must have neuroimaging to exclude a secondary cause of the neuralgia and a mean of >4 on the daily “average intensity”, on an 11-point Numerical Pain Rating Scale (0-10) during the 14-30 day screening period.

### Number of Subjects:

Approximately 90 patients are screened to randomize 60 patients in a 1:1 ratio into 2 treatment sequences receiving 75 mg rimegepant vs Placebo, using a 2-period, 2-sequence, crossover design.

### Objectives:

The primary objective of the study is to evaluate the efficacy of rimegepant compared to placebo in providing symptomatic pain relief in patients with refractory Trigeminal Neuralgia as measured by a 2-point or greater reduction in the average Numeric Pain Rating Scale between the two-week treatment phases.

### The Secondary Objectives of the Study are:

To assess the safety and tolerability of rimegepant relative to placebo in patients with Trigeminal Neuralgia
To evaluate the efficacy of rimegepant vs placebo for improving physical function in Trigeminal Neuralgia patients as measured by the Penn Facial Pain Scale-Revised
To evaluate the efficacy of rimegepant vs placebo for improving functional disability in Trigeminal Neuralgia patients as measured by the Pain Disability Index
To evaluate the efficacy of rimegepant vs placebo on global functioning as measured by the Patient Global Impression of Change Scale
To evaluate the efficacy of rimegepant vs placebo in providing symptomatic pain relief as captured by daily rating of worst pain episode as measured by the 11-point Numeric Pain Rating Scale
   The exploratory objectives of the study are:
To determine if there is a differential response of rimegepant for Trigeminal Neuralgia, purely paroxysmal (typical TN) versus Trigeminal Neuralgia with concomitant continuous pain (atypical TN)
To assess the efficacy of rimegepant vs placebo in providing symptomatic pain relief in patients with Trigeminal Neuralgia as measured by the use of daily rescue medications
To characterize the pharmacokinetics of rimegepant based on sparse sampling.

### Dose Selection Rationale:

In three positive, double-blind, placebo-controlled BHV-3000 studies, a 75 mg dose of rimegepant was demonstrated to be effective for the treatment of migraine. Additionally, based on preliminary data from an ongoing long-term safety study, rimegepant has been well tolerated at the 75 mg dose and demonstrates a consistent favorable safety profile.

### Study Design:

BHV3000-202 is a Phase 2, Multi-center, double-blind, placebo controlled, crossover trial assessing the safety, tolerability, and efficacy of rimegepant in treating Trigeminal Neuralgia in patients who failed to respond adequately to pharmacotherapy. Current inadequate response to therapy is defined by a daily pain score of greater than or equal to 4 on the “average intensity”, on the 11 point Numerical Pain Rating Scale during the 14 days prior to baseline. A crossover design with a placebo washout period of 7 days between treatments is employed due to the limited number of subjects, with each patient serving as their own control.

Prior to randomization, patients enter a 14-30 day screening period during which they complete daily self-reported assessments of pain intensity over a 24 hour period using an 11 point Numeric Pain Rating Scale, ranging from 0 (for no pain) to 10 (for the worst imaginable pain.) Throughout the study, patients are allowed to remain on their current medication regimens, provided they are on a stable dose for a minimum of 4 weeks prior to randomization visit.

Patients with a mean of ≥4 on the daily “average intensity” on the 11 point Numeric Pain Rating Scale, during the 14-30 day screening period, are randomized to one of two treatment sequences to receive rimegepant 75 mg administered orally vs placebo. Each sequence includes a 2 week treatment phase, with daily dosing of study drug or placebo. This is followed by a 7 day placebo washout period. After the placebo washout period, another 2 week treatment phase follows, again with once daily dosing of study drug or placebo. In the first sequence, patients receive placebo for the first treatment period and rimegepant 75 mg during the second treatment period. The second sequence has rimegepant 75 mg during the first 2 week treatment period, followed by placebo in the second treatment phase.

During each 2 week treatment phase and during the 7 day placebo washout period, patients complete the 11 point Numeric Pain Rating Scale daily, as described above. In addition, patients complete a paper diary daily to record efficacy data. The diary includes the daily recording of overall pain using the 11 point numeric pain rating scale, a recording of daily use of rescue medications, and a daily rating of worst pain episode using the 11 point Numeric Pain Rating Scale. This data is be entered into the case report form (CRF). Other secondary endpoints are assessed at the beginning and end of each treatment period, including the Penn Facial Pain Scale (a 12-item activities of daily living scale designed to specifically assess the impact of Trigeminal Neuralgia symptoms on daily activities), the Pain Disability Index (which measures the degree to which chronic pain interferes with 7 categories of daily activities, measured on a 11 point scale ranging from 0 (no disability) to 10 (worst disability). The Patient Global Impression of Change (PGIC: a patient self-reported global index scale) is assessed at the end of each treatment sequence. In addition, the Sheehan Suicidality Tracking Scale (STS) is administered as a safety measure at screening, as well as at every visit, as specified in the protocol.

All patients return to the study site within 14 days after the last day of dosing to complete a follow-up safety visit.

### Primary Endpoint:

Improvement of Trigeminal Neuralgia symptoms as assessed by the Numeric Pain Rating Scale over the 2-week treatment period

### Secondary Endpoints:

The frequency of unique subjects with adverse events, serious adverse events, adverse events leading to discontinuation, Sheehan Suicidality Tracking Scale (S-STS) total score and clinically significant laboratory and ECG test abnormalities, from case report forms and clinical laboratory evaluations
Penn Facial Pain Scale-Revised (Penn-FPS-R)
Pain Disability Index
Patient Global Impression of Change Scale (PGI-C)
Measurement of worst pain in a 24-hour period using the Numeric Pain Rating Scale

### Example 3—Oral Pharmaceutical Compositions for Treating Trigeminal Neuralgia

The CGRP receptor antagonists of one or more of a compound according to Formula I, Formula II, and/or Formula III can be formulated as an oral treatment for trigeminal neuralgia. Such oral treatments are expected to be useful for the treatment, prophylaxis or prevention of, halting the progression of, and/or reducing the risk of trigeminal neuralgia.

The oral pharmaceutical compositions of one or more of a compound according to Formula I, Formula II, and/or Formula III can be prepared in any appropriate oral dosage form, including but not limited to as pills, tablets including oral disintegrating tablets, or capsules. The pharmaceutical compositions may be formulated with one or more pharmaceutically acceptable excipients as disclosed herein or as known to a person of skill in the art.

In a study, the oral pharmaceutical compositions of one or more of a compound according to Formula I, Formula II, and/or Formula III can be administered to patients having trigeminal neuralgia using a dosing regimen described herein. The patients can be monitored for improvement of symptoms associated with trigeminal neuralgia. In some patients, treatment may continue beyond the treatment of trigeminal neuralgia to prophylactically treat, prevent, and/or prevent the risk of symptom recurrence. Subgroups of patients having a history of trigeminal neuralgia and symptom flares but no current or currently limited symptoms can be treated prophylactically with the expectation that further trigeminal neuralgia symptom flares can be reduced or avoided. As described herein, a treatment dose may differ in the amount of one or more of a compound according to Formula I, Formula II, and/or Formula III administered compared to a preventative dose, where the amount of one or more of Formula I, Formula II, and/or Formula III administered in the preventative dose may be, but is not necessarily, lower. The dosage amounts and particular dosage regimen applied to each patient can be decided upon by a medical professional using sound judgement according to the disclosure herein.

It is expected that such oral therapies can successfully treat trigeminal neuralgia and that, guided by these developed therapies, physicians can identify an effective course of trigeminal neuralgia treatment using the compounds, compositions, and methods of the present disclosure.

### Example 4—Topical Pharmaceutical Compositions for Treating Trigeminal Neuralgia

The CGRP receptor antagonists of one or more of a compound according to Formula I, Formula II, and/or Formula III can be formulated as a topical treatment for trigeminal neuralgia. Such topical treatments are expected to be useful for the treatment, prophylaxis or prevention of, halting the progression of, and/or reducing the risk of trigeminal neuralgia.

The topical pharmaceutical compositions of one or more of a compound according to Formula I, Formula II, and/or Formula III can be prepared in any appropriate dosage form, including but not limited to as lotions, creams, ointments, foams, patches, pastes, gels, suspensions, and solutions. The pharmaceutical compositions may be formulated with one or more pharmaceutically acceptable excipients as disclosed herein or as known to a person of skill in the art.

In a study, the topical pharmaceutical compositions of one or more of a compound according to Formula I, Formula II, and/or Formula III can be administered to patients having trigeminal neuralgia using a dosing regimen described herein. The patients can be monitored for improvement of symptoms associated with trigeminal neuralgia. In some patients, treatment may continue beyond the treatment of trigeminal neuralgia to prophylactically treat, prevent, and/or prevent the risk of symptom recurrence. Subgroups of patients having a history of trigeminal neuralgia and symptom flares but no current or currently limited symptoms can be treated prophylactically with the expectation that further trigeminal neuralgia symptom flares can be reduced or avoided. As described herein, a treatment dose may differ in the amount of one or more of a compound according to Formula I, Formula II, and/or Formula III administered compared to a preventative dose, where the amount of one or more of a compound according to Formula I, Formula II, and/or Formula III administered in the preventative dose may be, but is not necessarily, lower. The dosage amounts and particular dosage regimen applied to each patient can be decided upon by a medical professional using sound judgement according to the disclosure herein.

In some patients, topical pharmaceutical compositions can be applied directly onto, or adjacent to the trigeminal nerve or sites of frequent pain due to trigeminal neuralgia for treatment of active or recurrent trigeminal neuralgia symptoms. In some patients, topical pharmaceutical compositions can be applied as a preventative to regions of the head or face in which pain is frequently experienced.

It is expected that such topical therapies can successfully treat trigeminal neuralgia and that, guided by these developed therapies, physicians can identify an effective course of trigeminal neuralgia treatment using the compounds, compositions, and methods of the present disclosure.

### Example 5—Injectable Pharmaceutical Compositions for Treating Trigeminal Neuralgia

The CGRP receptor antagonists of one or more of a compound according to Formula I, Formula II, and/or Formula III can be formulated as an injectable treatment for trigeminal neuralgia. Such injectable treatments are expected to be useful for the treatment, prophylaxis or prevention of, halting the progression of, and/or reducing the risk of trigeminal neuralgia.

The injectable pharmaceutical compositions of one or more of a compound according to Formula I, Formula II, and/or Formula III can be prepared in any appropriate dosage form, including but not limited to as sterile solutions, suspensions, dispersions, or emulsions. The pharmaceutical compositions may be formulated with one or more pharmaceutically acceptable excipients as disclosed herein or as known to a person of skill in the art.

In a study, the injectable pharmaceutical compositions of one or more of a compound according to Formula I, Formula II, and/or Formula III can be administered to patients having trigeminal neuralgia using a dosing regimen described herein. The patients can be monitored for improvement of symptoms associated with trigeminal neuralgia. In some patients, treatment may continue beyond the treatment of trigeminal neuralgia to prophylactically treat, prevent, and/or prevent the risk of symptom recurrence. Subgroups of patients having a history of trigeminal neuralgia and symptom flares but no current or currently limited symptoms can be treated prophylactically with the expectation that further trigeminal neuralgia symptom flares can be reduced or avoided. As described herein, a treatment dose may differ in the amount of one or more of a compound according to Formula I, Formula II, and/or Formula III administered compared to a preventative dose, where the amount of one or more of a compound according to Formula I, Formula II, and/or Formula III administered in the preventative dose may be, but is not necessarily, lower. The dosage amounts and particular dosage regimen applied to each patient can be decided upon by a medical professional using sound judgement according to the disclosure herein.

In some patients, injectable pharmaceutical compositions can be injected intravenously, intramuscularly, subcutaneously, or via any other appropriate route for a systemic treatment of trigeminal neuralgia. In some patients, injectable pharmaceutical compositions can be injected locally at or adjacent to either the trigeminal nerve or a site of pain due to trigeminal neuralgia.

It is expected that such injectable therapies can successfully treat trigeminal neuralgia and that, guided by these developed therapies, physicians can identify an effective course of trigeminal neuralgia treatment using the compounds, compositions, and methods of the present disclosure.

### Example 6—Intranasal Pharmaceutical Compositions for Treating Trigeminal Neuralgia

The CGRP receptor antagonists of one or more of a compound according to Formula I, Formula II, and/or Formula III can be formulated as an intranasal treatment for trigeminal neuralgia. Intranasal pharmaceutical compositions are described generally in WO 2021/112707A1 published Jun. 24, 2021, which is incorporated by reference in its entirety herein. Such intranasal treatments are expected to be useful for the treatment, prophylaxis or prevention of, halting the progression of, and/or reducing the risk of trigeminal neuralgia.

The intranasal pharmaceutical compositions of one or more of a compound according to Formula I, Formula II, and/or Formula III can be prepared in any appropriate dosage form, including but not limited to as sterile solutions, saline solutions, isotonic solutions, buffered solutions, suspensions, dispersions, and emulsions. The pharmaceutical compositions may be formulated with one or more pharmaceutically acceptable excipients as disclosed herein or as known to a person of skill in the art.

In a study, the intranasal pharmaceutical compositions of one or more of a compound according to Formula I, Formula II, and/or Formula III can be administered to patients having trigeminal neuralgia using a dosing regimen described herein. The patients can be monitored for improvement of symptoms associated with trigeminal neuralgia. In some patients, treatment may continue beyond the treatment of trigeminal neuralgia to prophylactically treat, prevent, and/or prevent the risk of symptom recurrence. Subgroups of patients having a history of trigeminal neuralgia and symptom flares but no current or currently limited symptoms can be treated prophylactically with the expectation that further trigeminal neuralgia symptom flares can be reduced or avoided. As described herein, a treatment dose may differ in the amount of one or more of a compound according to Formula I, Formula II, and/or Formula III administered compared to a preventative dose, where the amount of one or more of a compound according to Formula I, Formula II, and/or Formula III administered in the preventative dose may be, but is not necessarily, lower. The dosage amounts and particular dosage regimen applied to each patient can be decided upon by a medical professional using sound judgement according to the disclosure herein.

It is expected that such intranasal therapies can successfully treat trigeminal neuralgia with varying degrees of success in different patients having different types of trigeminal neuralgia and trigeminal neuralgia symptoms, and that, guided by these developed therapies, physicians can identify an effective course of trigeminal neuralgia treatment using the compounds, compositions, and methods of the present disclosure.

### Example 7—Combination Treatments for Treating Trigeminal Neuralgia

Some patients having active trigeminal neuralgia symptoms can be treated in a study with one or more of an oral, topical, intranasal and/or injectable pharmaceutical composition according to Examples 3-6 simultaneously, alternatively, or initially via one route initially in a first treatment phase followed by a second route in a second treatment phase.

In a first subset of patients, a local topical treatment can be applied in a first treatment phase followed by an oral, intranasal, or injectable systemic treatment in a second treatment phase following the first. The local topical treatment, i.e. the first treatment phase, can be applied for a set period of time or until a change or improvement of symptoms is observed, after which a second treatment phase can be applied. The systemic oral, intranasal, or injectable treatment, i.e. the second treatment phase, would then be applied either i) until symptoms have been reduced or cleared and/or ii) for a prolonged period for prophylaxis, prevention, and reduction in the risk that the patient has further symptom flares.

In a second subset of patients, systemic oral, intranasal, or injectable treatment can be applied in a first treatment phase followed by a topical local treatment in a second treatment phase following the first. The systemic oral, intranasal, or injectable, i.e. the first treatment phase, can be applied for a set period of time or until a change or improvement of symptoms is observed, after which a second treatment phase can be applied. The local topical treatment, i.e. the second treatment phase, would then be applied either i) until symptoms have been reduced or cleared and/or ii) for a prolonged period for prophylaxis, prevention, and reduction in the risk that the patient has further symptom flares.

In a third subset of patients, systemic oral, intranasal, or injectable treatment can be applied concurrently with a topical local treatment. Within the subset, after a set period of time or after improvement of symptoms is observed, some patients can discontinue the local topical treatment and continue receiving the systemic oral, intranasal, or injectable treatment either i) until symptoms have been reduced or cleared and/or ii) for a prolonged period for prophylaxis, prevention, and reduction in the risk that the patient has further symptom flares. For other patients within the subset, after a set period of time or after improvement of symptoms is observed, some patients can discontinue the oral, intranasal, or injectable systemic treatment and continue receiving the local topical treatment either i) until symptoms have been reduced or cleared and/or ii) for a prolonged period for prophylaxis, prevention, and reduction in the risk that the patient has further symptom flares.

It is expected that such combination therapies can successfully treat trigeminal neuralgia and that, guided by these developed therapies, physicians can identify an effective course of trigeminal neuralgia treatment using the compounds, compositions, and methods of the present disclosure.

### INCORPORATION BY REFERENCE

The entire disclosure of each of the patent documents, including certificates of correction, patent application documents, scientific articles, governmental reports, websites, and other references referred to herein is incorporated by reference herein in its entirety for all purposes. In case of a conflict in terminology, the present specification controls.

### EQUIVALENTS

The invention can be embodied in other specific forms without departing from the spirit or essential characteristics thereof. The foregoing embodiments are to be considered in all respects illustrative rather than limiting on the invention described herein. In the various embodiments of the present invention, where the term comprises is used with respect to the recited components or methods, it is also contemplated that the invention consists essentially of, or consists of, the recited components or methods. Furthermore, it should be understood that the order of steps or order for performing certain actions is immaterial so long as the invention remains operable. Moreover, two or more steps or actions can be conducted simultaneously.

In the specification, the singular forms also include the plural forms, unless the context clearly dictates otherwise. Unless defined otherwise, all technical and scientific terms used herein have the same meaning as commonly understood by one of ordinary skill in the art to which this invention belongs. In the case of conflict, the present specification will control.

All percentages and ratios used herein, unless otherwise indicated, are by weight. It is recognized the mass of an object is often referred to as its weight in everyday usage and for most common scientific purposes, but that mass technically refers to the amount of matter of an object, whereas weight refers to the force experienced by an object due to gravity. Also, in common usage the “weight” (mass) of an object is what one determines when one “weighs” (masses) an object on a scale or balance.

1-60. (canceled)

61. A method of treating, ameliorating, alleviating, providing for prophylaxis or prevention of, halting the progression of, and/or reducing the risk of trigeminal neuralgia, the method comprising administering to a subject in need thereof a CGRP receptor antagonist wherein the CGRP receptor antagonist is the compound (R)-N-(3-(7-methyl-1H-indazol-5-yl)-1-(4-(1-methylpiperidin-4-yl)piperazin-1-yl)-1-oxopropan-2-yl)-4-(2-oxo-1,2-dihydroquinolin-3-yl)piperidine-1-carboxamide, which has the following structure: 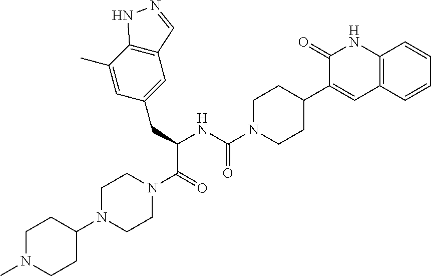 [SMILES: Cc1cc(C[C@@H](NC(=O)N2CCC(c3cc4ccccc4[nH]c3=O)CC2)C(=O)N2CCN(C3CCN(C)CC3)CC2)cc2cn[nH]c12]
or a pharmaceutically acceptable salt thereof.

62. The method of claim 61, wherein the CGRP antagonist is administered in a unit dosage selected from the group consisting of about 10 mg, about 20 mg, about 30 mg, about 40 mg, about 50 mg, about 60 mg, about 70 mg, about 75 mg, about 80 mg, about 100 mg, about 120 mg, about 140 mg, about 160 mg, about 180 mg, or about 200 mg.

63. The method of claim 61, wherein the CGRP antagonist is administered in a dosage of 0.01-100 mg/kg body weight.

64. The method of claim 61, wherein the CGRP antagonist is administered in an amount corresponding to an area under curve (AUC) within about 80-125% of about 25, 50, 100, 150, 200, 250, 300, 400, 500, 600, 700, 800, 900, 1000, 1300, 1600, 1900, 2500, 3500, 5000, 7500, 10,000, 15,000, 20,000, 30,000, 50,000, or 100,000 hr*ng/mL.

65. The method of claim 61, wherein the CGRP antagonist is administered in an amount corresponding to a Cₘₐₓ value within about 80-125% of about 1, 2, 3, 4, 5, 8, 10, 20, 35, 50, 80, 100, 150, 200, 250, 300, 400, 500, 600, 700, 800, 900, 1000, 1300, 1600, 1900, 2500, 3500, 5000, 7500, 10,000, 15,000, 20,000, 30,000, 50,000, and/or 100,000 ng/mL.

66. The method of claim 65, wherein the CGRP antagonist is administered in an amount corresponding to a Cₘₐₓ/AUC ratio within about 80-125% of about 0.01, 0.03, 0.05, 0.08, 0.1, 0.13, 0.17, 0.2, 0.25, 0.3, 0.35, 0.4, 0.45, 0.5, 0.6, 0.7, 0.8, or 0.9.

67. The method of a claim 61, wherein the CGRP antagonist is administered at an interval of daily, twice daily, three times daily, four times daily, once every other day, once every two days, once every three days, once every four days, once every five days, once every six days, or once weekly.

68. The method of a claim 61, wherein the CGRP antagonist is administered by a route selected from oral, intrabuccal, intranasal, inhalation, parenteral, intravenous, topical, injectable and/or transdermal.

69. The method of claim 61, wherein the CGRP antagonist is administered in a unit dosage of 5 mg, 10 mg, or 20 mg.

70. The method of claim 69, wherein the CGRP antagonist is administered intranasally.

71-97. (canceled)

98. The method of claim 61, the method comprising administering to a subject in need thereof a pharmaceutical composition comprising the CGRP receptor antagonist and one or more pharmaceutically acceptable carriers.

99. The method of claim 98, wherein the composition is formulated for oral administration.

100. The method of claim 98, wherein the composition is formulated for injectable administration.

101. The method of claim 98, wherein the composition is formulated for topical administration.

102. The method of claim 61, wherein the trigeminal neuralgia is trigeminal neuralgia type 1 (TN1), trigeminal neuralgia type 2 (TN2), or both TN1 and TN2.

103. The method of claim 61, wherein the treatment is characterized by an improvement in the Numeric Pain Rating Scale of at least 2 points compared to the patient baseline absent treatment.

104-106. (canceled)
